(19) United States
(12) Patent Application Publication
(10) Pub. No.: US 2024/0034726 A9
(43) Pub. Date: Feb. 1, 2024

(54) MAP KINASE KINASE (MKK7) INHIBITORS AND USES THEREOF

(71) Applicant: Dana-Farber Cancer Institute, Inc., Boston, MA (US)

(72) Inventors: Nathanael S. Gray, Stanford, CA (US); Steven P. Treon, Jamaica Plain, MA (US); Jinhua Wang, Winchester, MA (US); Li Tan, Pudong, Shanghai (CN); Guang Yang, Natick, MA (US)

(73) Assignee: Dana-Farber Cancer Institute, Inc., Boston, MA (US)

(21) Appl. No.: 17/802,896

(22) PCT Filed: Feb. 25, 2021
(86) PCT No.: PCT/US2021/019602

§ 371 Date: Aug. 26, 2022

Related U.S. Application Data
(60) Provisional application No. 62/983,330, filed on Feb. 28, 2020.

(51) Int. Cl. C07D 403/12; C07D 403/14

(52) U.S. Cl. CPC C07D 403/12; C07D 403/14

(57) ABSTRACT

Provided herein are compounds of Formula (I), and pharmaceutically acceptable salts, solvates, hydrates, polymorphs, co-crystals, tautomers, stereoisomers, isotopically labeled derivatives, prodrugs, and compositions thereof. Also provided are methods and kits involving the disclosed compounds or compositions for treating and/or preventing diseases (e.g., inflammatory diseases, neurological diseases (e.g., Alzheimer's disease), infectious diseases (e.g., viral infections (e.g., infections caused by viral human hepatitis (e.g., hepatitis B), a flaviviridae family virus (e.g., a flavivims (e.g., Zika virus)))), and proliferative diseases (e.g., cancer (e.g., lung cancer, prostate cancer, skin cancer, thyroid cancer, pancreatic cancer, colorectal cancer, liver cancer, leukemia, or lymphoma)) in a subject. Provided are methods of inhibiting a protein kinase (e.g., MAP2K (e.g., MKK7)) in a subject. Also provided are methods of inhibiting MKK7 in a subject in need thereof.

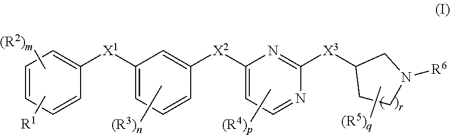

### RELATED APPLICATIONS

The present application claims priority under 35 U.S.C. § 119(e) to U.S. provisional application, U.S. Ser. No. 62/983,330, filed Feb. 28, 2020, which is incorporated herein by reference.

### BACKGROUND OF THE INVENTION

Mitogen-activated protein kinase kinase 7 (MKK7, MEK7, MAP2K7) is a member of mitogen-activated kinase kinase (MAP2K) subfamily involving in the c-Jun N-terminal kinase (JNK) signaling pathway. Targeting the MKK7 pathway may achieve selective shutdown of JNK signaling under pathway stimulation, while still allowing for some basal JNK activity to avoid potential systemic toxicity. Activation of this pathway is implicated in inflammatory diseases, Alzheimer's disease, Zika virus, Hepatitis B infection, and cancer.

### SUMMARY OF THE INVENTION

Described herein are compounds of Formula (I), and pharmaceutically acceptable salts, solvates, hydrates, polymorphs, co-crystals, tautomers, stereoisomers, isotopically labeled derivatives, prodrugs, and compositions thereof. The compounds of Formula (I) described herein may inhibit the activity of a protein kinase (e.g., MAP2K (e.g., MKK7))) in a subject, biological sample, or cell. The compounds disclosed in this application are useful as therapeutics as well as for studying the JNK signaling pathway.

Also disclosed herein are methods of using the disclosed compounds, and pharmaceutically acceptable salts, solvates, hydrates, polymorphs, co-crystals, tautomers, stereoisomers, isotopically labeled derivatives, prodrugs, and compositions thereof, to study the inhibition of a protein kinase (e.g., MAP2K (e.g., MKK7)). The compounds described herein may be useful as therapeutics for the prevention and/or treatment of diseases associated with the overexpression and/or aberrant (e.g., increased or unwanted) activity of a protein kinase (e.g., MAP2K (e.g., MKK7)). The compounds described herein may also be useful in treating and/or preventing a variety of diseases and conditions, such as, an inflammatory disease, neurological disease (e.g., Alzheimer's disease), infectious disease (e.g., viral infections (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)), in a subject in need thereof. Also provided are uses, pharmaceutical compositions, and kits including a compound described herein.

In one aspect, the present disclosure provides a compound of Formula (I):

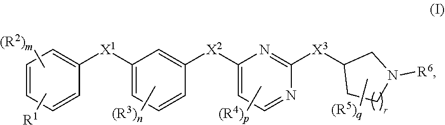

or a pharmaceutically acceptable salt, solvate, hydrate, polymorph, co-crystal, tautomer, stereoisomer, or prodrug thereof, wherein:

R¹ is substituted or unsubstituted C₁-C₆ alkyl;

R², R³, R⁴, and R⁵ are each independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl, —OR^{a}, —N(R^{a})₂, —SR^{a}, —C(═O)R^{a}, —C(═O)OR^{a}, —OC(═O)R^{a}, —C(═O)N(R^{a})₂, —N(R^{a})C(═O)R^{a}, —CN, —SCN, or —NO₂;

R⁶ is substituted or unsubstituted alkyl, or substituted or unsubstituted acyl;

X¹ is —O—, —N(R^{b})—, —S—, —C(═O)—, —C(═O)N(R^{b})—, or —N(R^{b})C(═O)—;

X² and X³ are each independently —O—, —N(R^{c})—, or —S—;

each instance of R^{a}, R^{b}, and R^{c} is independently hydrogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;

m, n, p, and q are each independently 0, 1, 2, 3, or 4; and

r is 1, or 2.

Exemplary compounds of Formula (I) include, but are not limited to:

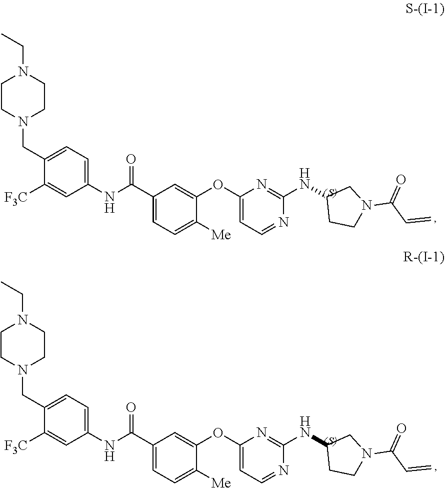

or a pharmaceutically acceptable salt, solvate, hydrate, polymorph, co-crystal, tautomer, stereoisomer, isotopically labeled derivative, or prodrug thereof.

In another aspect, the present disclosure provides pharmaceutical compositions including a compound described herein, and optionally a pharmaceutically acceptable excipient. In certain embodiments, the pharmaceutical compositions described herein include a therapeutically or prophylactically effective amount of a compound described herein. The pharmaceutical composition may be useful for treating and/or preventing a disease (e.g., an inflammatory disease, neurological disease (e.g., Alzheimer's disease), infectious disease (e.g., viral infections (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma))) in a subject in need thereof. The pharmaceutical composition may be useful for inhibiting the activity of a protein kinase (e.g., MAP2K (e.g., MKK7)) in a subject, biological sample, or cell. The compounds disclosed herein may be useful in treating and/or preventing a disease or condition, e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative diseases (e.g., cancer (e.g., lung cancer, prostate cancer, skin cancer, thyroid cancer, pancreatic cancer, colorectal cancer, liver cancer, leukemia, or lymphoma)).

In another aspect, the present disclosure provides pharmaceutical compositions including a compound described herein, and optionally a pharmaceutically acceptable excipient. In certain embodiments, the pharmaceutical compositions described herein include a therapeutically or prophylactically effective amount of a compound described herein. The pharmaceutical composition may be useful for treating a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma))) in a subject in need thereof, or inhibiting the activity of a protein kinase (e.g., MAP2K (e.g., MKK7)) in a subject, biological sample, or cell.

In another aspect, described herein are methods for treating and/or preventing a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma))).

Another aspect relates to methods of inhibiting the activity of a protein kinase (e.g., MAP2K (e.g., MKK7)) using a compound described herein in a biological sample or cell. In another aspect, described herein are methods of inhibiting the activity of a protein kinase (e.g., MAP2K (e.g., MKK7)) using a compound described herein in a subject. In certain aspects, the method involves the inhibition of MAP2K (e.g., MKK7)). In certain aspects, the method involves the inhibition of MKK7.

Described herein are methods for administering to a subject in need thereof a compound, or pharmaceutical composition thereof, as described herein, in an amount effective for treating a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma))) in a subject in need thereof. In certain embodiments, a method described herein further includes administering to the subject an additional pharmaceutical agent. Also described are methods of contacting a cell with a compound, or pharmaceutical composition thereof, as described herein, in an amount effective for inhibiting the activity of a protein kinase (e.g., MAP2K (e.g., MKK7)) in the cell.

In yet another aspect, the present disclosure provides compounds of Formula (I), and pharmaceutically acceptable salts, solvates, hydrates, polymorphs, co-crystals, tautomers, stereoisomers, isotopically labeled derivatives, prodrugs, and compositions thereof, for use in the treatment of a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma))) in a subject.

Another aspect of the present disclosure relates to kits comprising a container with a compound, or pharmaceutical composition thereof, as described herein. The kits described herein may include a single dose or multiple doses of the compound or pharmaceutical composition. The kits may be useful in a method of the disclosure. In certain embodiments, the kit further includes instructions for using the compound or pharmaceutical composition. A kit described herein may also include information (e.g. prescribing information) as required by a regulatory agency, such as the U.S. Food and Drug Administration (FDA).

The details of one or more embodiments of the invention are set forth herein. Other features, objects, and advantages of the invention will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modem Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

In a formula the bond [symbol] is a single bond, the dashed line - - - is a single bond or absent, and the bond [symbol] or [symbol] is a single or double bond.

Unless otherwise provided, a formula includes compounds that do not include isotopically enriched atoms and also compounds that include isotopically enriched atoms. Compounds that include isotopically enriched atoms may be useful, for example, as analytical tools and/or probes in biological assays.

When a range of values (“range”) is listed, it is intended to encompass each value and sub-range within the range. A range is inclusive of the values at the two ends of the range unless otherwise provided. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

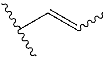

may be in the (E)- or (Z)-configuration.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₋₂), cyclododecenyl (C₁₋₂), cyclotridecane (C₁₋₃), cyclotetradecane (C₁₋₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{aa})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{b} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}a, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₁₀ alkyl, C₁₋₁₀ perhaloalkyl, C₁₋₁₀ alkenyl, C₁₋₁₀alkynyl, heteroC₁₋₁₀alkyl, heteroC₁₋₁₀alkenyl, heteroC₁₋₁₀alkynyl, C₃₋₁₀carbocyclyl, C₆₋₁₀aryl, 3-10membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁻X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₄ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₄ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀alkenyl, C₁₋₁₀alkynyl, heteroC₁₋₁₀ alkyl, heteroC₁₋₁₀ alkenyl, heteroC₁₋₁₀ alkynyl, C₃₋₁₀carbocyclyl, C₆₋₁₀ aryl, 3-10 membered heterocyclyl, or 5-10 membered heteroaryl; or two geminal R^{gg} substituents can be joined to form ═O or ═S; and

each X⁻ is a counterion.

In certain embodiments, the carbon atom substituents are independently halogen, substituted (e.g., substituted with one or more halogen) or unsubstituted C1-6 alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₁₀alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each Rbb is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})2, —CN, —SCN, or —NO₂. In certain embodiments, the carbon atom substituents are independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{aa})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on the nitrogen atom is an nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., the moieties that include the nitrogen atom to which the nitrogen protecting groups (e.g., —C(═O)R^{aa})) are directly attached) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., the moieties that include the nitrogen atom to which the nitrogen protecting groups (e.g., —C(═O)OR^{aa}) are directly attached) include methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., the moieties that include the nitrogen atom to which the nitrogen protecting groups (e.g., —S(═O)₂R^{aa}) are directly attached) include p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the nitrogen protecting groups include Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{aa})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “heteroatom” refers to an atom that is not hydrogen or carbon. In certain embodiments, the heteroatom is nitrogen. In certain embodiments, the heteroatom is oxygen. In certain embodiments, the heteroatom is sulfur.

These and other exemplary substituents are described in more detail in the Detailed Description, Examples, and Claims. This disclosure is not intended to be limited in any manner by the above exemplary listing of substituents.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The term “co-crystal” refers to a crystalline structure comprising at least two different components (e.g., a compound of Formula (I) and an acid), wherein each of the components is independently an atom, ion, or molecule. In certain embodiments, none of the components is a solvent. In certain embodiments, at least one of the components is a solvent. A co-crystal of a compound Formula (I) and an acid is different from a salt formed from a compound of Formula (I) and the acid. In the salt, a compound of Formula (I) is complexed with the acid in a way that proton transfer (e.g., a complete proton transfer) from the acid to a compound of Formula (I) easily occurs at room temperature. In the co-crystal, however, a compound of Formula (I) is complexed with the acid in a way that proton transfer from the acid to a compound of Formula (I) does not easily occur at room temperature. In certain embodiments, in the co-crystal, there is no proton transfer from the acid to the compound of Formula (I). In certain embodiments, in the co-crystal, there is partial proton transfer from the acid to the compound of Formula (I). Co-crystals may be useful to improve the properties (e.g., solubility, stability, and ease of formulation) of a compound of Formula (I).

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting the activity of a protein kinase (e.g., MAP2K (e.g., MKK7)) in a subject, biological sample, or cell. In certain embodiments, a therapeutically effective amount is an amount sufficient for treating a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma))) in a subject in need thereof.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting the activity of a protein kinase (e.g., MAP2K (e.g., MKK7)) in a subject, biological sample, or cell. In certain embodiments, a prophylactically effective amount is an amount sufficient for treating a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma))) in a subject in need thereof.

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

An “infectious disease” refers to an infection with a microorganism, such as a fungus, bacteria, or virus. In certain embodiments, the microbial infection is an infection with a fungus, i.e., a fungal infection. In certain embodiments, the microbial infection is an infection with a virus, i.e., a viral infection. In certain embodiments, viral infections caused by viral human hepatitis (e.g., hepatitis B). In certain embodiments, viral infections are caused by a flaviviridae family virus (e.g., a flavivirus (e.g., Zika virus)). In certain embodiments, the microbial infection is an infection with a bacteria, i.e., a bacterial infection. Various microbial infections include, but are not limited to, skin infections, GI infections, urinary tract infections, genito-urinary infections, sepsis, blood infections, and systemic infections.

### DETAILED DESCRIPTION OF CERTAIN EMBODIMENTS OF THE INVENTION

Mitogen-activated protein kinase kinase 7 (MKK7) is a member of the mitogen-activated kinase kinase (MAP2K) subfamily and is implicated in many diseases, including, but not limited to, inflammatory diseases, neurological diseases (e.g., Alzheimer's disease), infectious diseases (e.g., viral infections (e.g., infections caused by viral human hepatitis (e.g., hepatitis B), a flaviviridae family virus (e.g., a flavivirus (e.g., Zika virus)))), and proliferative diseases (e.g., cancer (e.g., lung cancer, prostate cancer, skin cancer, thyroid cancer, pancreatic cancer, colorectal cancer, liver cancer, leukemia, or lymphoma)). Targeting the MKK7 pathway may achieve selective shutdown of JNK signaling under pathway stimulation, while still allowing for some basal JNK activity to avoid potential systemic toxicity.

Provided herein are compounds of Formula (I), and pharmaceutically acceptable salts, solvates, hydrates, polymorphs, co-crystals, tautomers, stereoisomers, isotopically labeled derivatives, and prodrugs thereof. The disclosed compounds may be kinase inhibitors. In certain embodiments, the kinase being targeted is a Mitogen-activated protein kinase kinase (MAP2K). In certain embodiments, the kinase being targeted is mitogen-activated protein kinase kinase 7 (i.e., MKK7, MEK7, MAP2K7). In certain embodiments, the MAP2K is MKK7. Also disclosed is pharmaceutical compositions and kits comprising the disclosed compounds.

Without wishing to be bound by any theory, inhibition of MKK7 by the compounds described herein may achieve selective shutdown of JNK signaling pathway, while still allowing for some basal JNK activity to avoid potential systemic toxicity. In certain embodiments, the inhibition of a MAP2K (e.g., MKK7) by the compounds described herein may selectively inhibit the activity of a JNK (e.g., JNK1, JNK2). In certain embodiments, the inhibition of a MAP2K (e.g., MKK7) by the compounds described herein may selectively inhibit the activity of JNK1. In certain embodiments, the inhibition of a MAP2K (e.g., MKK7) by the compounds described herein may selectively inhibit the activity of JNK2.

Further provided are methods of using the disclosed compounds, pharmaceutical compositions, and kits for treating a disease in a subject in need thereof. In certain embodiments, the disease is an inflammatory disease. In certain embodiments, the disease is a neurological disease (e.g., Alzheimer's disease). In certain embodiments, the disease is an infectious disease (e.g., a viral infection (e.g., an infection caused by viral human hepatitis (e.g., hepatitis B), or a flaviviridae family virus (e.g., a flavivirus (e.g., Zika virus)))). In certain embodiments, the disease is a proliferative disease (e.g., cancer (e.g., lung cancer, prostate cancer, skin cancer, thyroid cancer, pancreatic cancer, colorectal cancer, liver cancer, leukemia, or lymphoma)). In certain embodiments, the disease is associated with the overexpression and/or aberrant (e.g., increased or unwanted) activity of a protein kinase (e.g., MAP2K (e.g., MKK7)). Further provided are methods of using the disclosed compounds, pharmaceutical compositions, and kits for inhibiting the activity of a kinase (e.g., MAP2K (e.g., MKK7)) in a subject in need thereof or in a biological sample or cell.

### Compounds

In certain embodiments, a compound described herein is of the Formula (I):

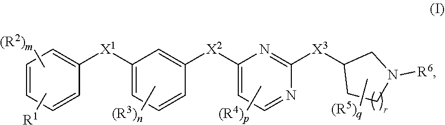

or a pharmaceutically acceptable salt, solvate, hydrate, polymorph, co-crystal, tautomer, stereoisomer, or prodrug thereof, wherein:

R¹ is substituted or unsubstituted C₁-C₆ alkyl;

R², R³, R⁴, and R⁵ are each independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl, —OR^{a}, —N(R^{a})₂, —SR^{a}, —C(═O)R^{a}, —C(═O)OR^{a}, —OC(═O)R^{a}, —C(═O)N(R^{a})₂, —N(R^{a})C(═O)R^{a}, —CN, —SCN, —NO₂;

R⁶ is substituted or unsubstituted alkyl, or substituted or unsubstituted acyl;

X¹ is —O—, —N(R^{b})—, —S—, —C(═O)—, —C(═O)N(R^{b})—, or —N(R^{b})C(═O)—;

X² and X³ are each independently —O—, —N(R^{c})—, or —S—;

each instance of R^{a}, R^{b}, and R^{c} is independently hydrogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom;

m, n, p, and q are each independently 0, 1, 2, 3, or 4; and

r is 1, or 2.

Formula (I) includes the substituent R¹. In certain embodiments, R¹ is substituted or unsubstituted C₁-C₆ alkyl. In certain embodiments, R¹ is substituted or unsubstituted C₁-C₆ alkyl. In certain embodiments, R¹ is C₁-C₆ haloalkyl. In certain embodiments, R¹ is —CH₃. In certain embodiments, R¹ is —CHF₂. In certain embodiments, R¹ is —CH₂F. In certain embodiments, R¹ is —CF₃.

Formula (I) includes the substituents R², R³, R⁴, and R⁵. In certain embodiments, R², R³, R⁴, and R⁵ are each independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl, —OR^{a}, —N(R^{a})₂, —SR^{a}, —C(═O)R^{a}, —C(═O)OR^{a}, —OC(═O)R^{a}, —C(═O)N(R^{a})₂, —N(R^{a})C(═O)R^{a}, —CN, —SCN, —NO₂.

In certain embodiments, R², R³, R⁴, or R⁵ includes the substituent R^{a}. In certain embodiments, R^{a} is hydrogen. In certain embodiments, R^{a} is substituted or unsubstituted alkyl. In certain embodiments, R^{a} is substituted or unsubstituted alkenyl. In certain embodiments, R^{a} is substituted or unsubstituted alkynyl. In certain embodiments, R^{a} is substituted or unsubstituted acyl. In certain embodiments, R^{a} is substituted or unsubstituted carbocyclyl. In certain embodiments, R^{a} is substituted or unsubstituted heterocyclyl. In certain embodiments, R^{a} is substituted or unsubstituted aryl. In certain embodiments, R^{a} is substituted or unsubstituted heteroaryl. In certain embodiments, when attached to a nitrogen atom, R^{a} is a nitrogen protecting group. In certain embodiments, when attached to an oxygen atom, R^{a} is an oxygen protecting group. In certain embodiments, when attached to a sulfur atom, R^{a} is a sulfur protecting group.

In certain embodiments, m, n, p, and q are each independently 0, 1, 2, 3, or 4.

In certain embodiments, m is 0, 1, 2, 3, or 4. In certain embodiments, m is 0. In certain embodiments, m is 1. In certain embodiments, m is 2. In certain embodiments, m is 3. In certain embodiments, m is 4.

In certain embodiments, n is 0, 1, 2, 3, or 4. In certain embodiments, n is 0. In certain embodiments, n is 1. In certain embodiments, n is 2. In certain embodiments, n is 3. In certain embodiments, n is 4.

In certain embodiments, p is 0, 1, 2, 3, or 4. In certain embodiments, p is 0. In certain embodiments, p is 1. In certain embodiments, p is 2. In certain embodiments, p is 3. In certain embodiments, p is 4.

In certain embodiments, q is 0, 1, 2, 3, or 4. In certain embodiments, q is 0. In certain embodiments, q is 1. In certain embodiments, q is 2. In certain embodiments, q is 3. In certain embodiments, q is 4.

In certain embodiments, r is 1 or 2. In certain embodiments, r is 1. In certain embodiments, r is 2.

In certain embodiments, R² is substituted or unsubstituted alkyl, substituted or unsubstituted heterocyclyl, or substituted or unsubstituted heteroaryl.

In certain embodiments, R² is substituted or unsubstituted heterocyclyl. In certain embodiments, R² is a substituted or unsubstituted piperazine.

In certain embodiments, R² is of the formula:

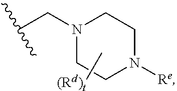

wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;

R^{e} is independently hydrogen, substituted or unsubstituted C₁-C₆ alkyl, substituted or unsubstituted acyl, or a nitrogen protecting group; and

t is 0, 1, 2, 3, 4, 5, 6, 7, or 8.

In certain embodiments, R² is of the formula:

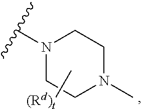

wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl; and

t is 0, 1, 2, 3, 4, 5, 6, 7, or 8.

In certain embodiments, R² is of the formula:

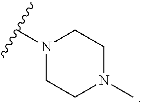 [SMILES: *N1CCN(C)CC1]

In certain embodiments, R² is substituted or unsubstituted alkyl.

In certain embodiments, R² is of the formula:

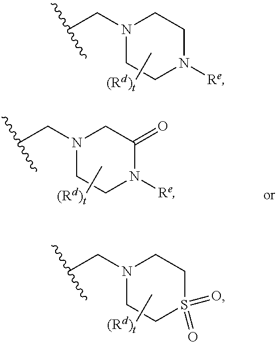

wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;

R^{e} is independently hydrogen, substituted or unsubstituted C₁-C₆ alkyl, substituted or unsubstituted acyl, or a nitrogen protecting group; and

t is 0, 1, 2, 3, 4, 5, 6, 7, or 8.

In certain embodiments, R² is of the formula:

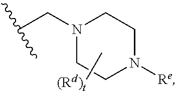

wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;

R^{e} is independently hydrogen, substituted or unsubstituted C₁-C₆ alkyl, substituted or unsubstituted acyl, or a nitrogen protecting group; and

t is 0, 1, 2, 3, 4, 5, 6, 7, or 8.

In certain embodiments, R² is of the formula:

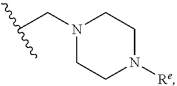

wherein R^{e} is independently hydrogen, substituted or unsubstituted C₁-C₆ alkyl, substituted or unsubstituted acyl, or a nitrogen protecting group.

In certain embodiments, R² is of the formula:

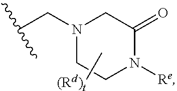

wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;

R^{e} is independently hydrogen, substituted or unsubstituted C₁-C₆ alkyl, substituted or unsubstituted acyl, or a nitrogen protecting group; and

t is 0, 1, 2, 3, 4, 5, 6, 7, or 8.

In certain embodiments, R² is of the formula:

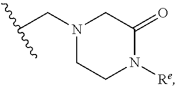

wherein R^{e} is independently hydrogen, substituted or unsubstituted C₁-C₆ alkyl, substituted or unsubstituted acyl, or a nitrogen protecting group.

In certain embodiments, R² is of the formula:

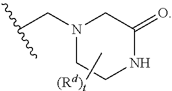

In certain embodiments, R² is of the formula:

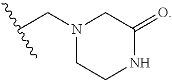 [SMILES: *CN1CCNC(=O)C1]

In certain embodiments, R² is of the formula:

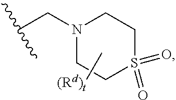

wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl; and

t is 0, 1, 2, 3, 4, 5, 6, 7, or 8.

In certain embodiments, R² is of the formula:

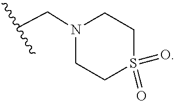 [SMILES: *CN1CCS(=O)(=O)CC1]

In certain embodiments, R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl. In certain embodiments, R^{d} is hydrogen. In certain embodiments, R^{d} is halogen. In certain embodiments, R^{d} is substituted or unsubstituted alkyl. In certain embodiments, R^{d} is substituted or unsubstituted alkenyl. In certain embodiments, R^{d} is substituted or unsubstituted alkynyl. In certain embodiments, R^{d} is substituted or unsubstituted acyl. In certain embodiments, R^{d} is substituted or unsubstituted carbocyclyl. In certain embodiments, R^{d} is substituted or unsubstituted heterocyclyl. In certain embodiments, R^{d} is substituted or unsubstituted aryl. In certain embodiments, R^{d} is substituted or unsubstituted heteroaryl.

In certain embodiments, R^{e} is independently hydrogen, substituted or unsubstituted C₁-C₆ alkyl, substituted or unsubstituted acyl, or a nitrogen protecting group. In certain embodiments, R^{e} is hydrogen. In certain embodiments, R^{e} is substituted or unsubstituted C₁-C₆ alkyl. In certain embodiments, R^{e} is substituted or unsubstituted acyl. In certain embodiments, R^{e} is unsubstituted C₁-C₆ alkyl. In certain embodiments, R^{e} is unsubstituted acyl. In certain embodiments, R^{e} is substituted C₁-C₆ alkyl. In certain embodiments, R^{e} is substituted acyl. In certain embodiments, R^{e} is a nitrogen protecting group. In certain embodiments, R^{e} is H. In certain embodiments, R^{e} is substituted or unsubstituted C₁-C₆ alkyl. In certain embodiments, R^{e} is methyl. In certain embodiments, R^{e} is ethyl.

In certain embodiments, R² is substituted or unsubstituted heteroaryl. In certain embodiments, R² is of the formula:

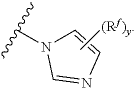

wherein R^{f} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl; and

y is 0, 1, 2, or 3.

In certain embodiments, R² is of the formula:

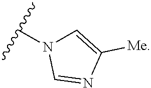 [SMILES: *n1cnc(C)c1]

In certain embodiments, R^{f} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl. In certain embodiments, R^{f} is hydrogen. In certain embodiments, R^{f} is halogen. In certain embodiments, R^{f} is substituted or unsubstituted alkyl. In certain embodiments, R^{f} is substituted or unsubstituted methyl. In certain embodiments, R^{f} is unsubstituted methyl. In certain embodiments, R^{f} is substituted or unsubstituted alkenyl. In certain embodiments, R^{f} is substituted or unsubstituted alkynyl. In certain embodiments, R^{f} is substituted or unsubstituted acyl. In certain embodiments, R^{f} is substituted or unsubstituted carbocyclyl. In certain embodiments, R^{f} is substituted or unsubstituted heterocyclyl. In certain embodiments, R^{f} is substituted or unsubstituted aryl. In certain embodiments, R^{f} is substituted or unsubstituted heteroaryl.

In certain embodiments, t is 0, 1, 2, 3, 4, 5, 6, 7, or 8. In certain embodiments, t is 0. In certain embodiments, t is 1. In certain embodiments, t is 2. In certain embodiments, t is 3. In certain embodiments, t is 4. In certain embodiments, t is 5. In certain embodiments, t is 6. In certain embodiments, t is 7. In certain embodiments, t is 8.

In certain embodiments, y is 0, 1, 2, or 3. In certain embodiments, y is 0. In certain embodiments, y is 1. In certain embodiments, y is 2. In certain embodiments, y is 3.

In certain embodiments, R³ is hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl, —OR^{a}, —N(R^{a})₂, —SR^{a}, —C(═O)R^{a}, —C(═O)OR^{a}, —OC(═O)R^{a}, —C(═O)N(R^{a})₂, —N(R^{a})C(═O)R^{a}, —CN, —SCN, —NO₂. In certain embodiments, R³ is substituted or unsubstituted alkyl. In certain embodiments, R³ is unsubstituted alkyl. In certain embodiments, R³ is substituted alkyl. In certain embodiments, R³ is substituted or unsubstituted C₁-C₆ alkyl. In certain embodiments, R³ is unsubstituted C₁-C₆ alkyl. In certain embodiments, R³ is substituted or unsubstituted methyl. In certain embodiments, R³ is unsubstituted methyl.

In certain embodiments, R⁴ is hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl, —OR^{a}, —N(R^{a})₂, —SR^{a}, —C(═O)R^{a}, —C(═O)OR^{a}, —OC(═O)R^{a}, —C(═O)N(R^{a})₂, —N(R^{a})C(═O)R^{a}, —CN, —SCN, —NO₂. In certain embodiments, R⁴ is substituted or unsubstituted alkyl. In certain embodiments, R⁴ is unsubstituted alkyl. In certain embodiments, R⁴ is substituted alkyl. In certain embodiments, R⁴ is substituted or unsubstituted C₁-C₆ alkyl. In certain embodiments, R⁴ is unsubstituted C₁-C₆ alkyl.

In certain embodiments, R⁵ is hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl, —OR^{a}, —N(R^{a})₂, —SR^{a}, —C(═O)R^{a}, —C(═O)OR^{a}, —OC(═O)R^{a}, —C(═O)N(R^{a})₂, —N(R^{a})C(═O)R^{a}, —CN, —SCN, —NO₂. In certain embodiments, R⁵ is substituted or unsubstituted alkyl. In certain embodiments, R⁵ is unsubstituted alkyl. In certain embodiments, R⁵ is substituted alkyl. In certain embodiments, R⁵ is substituted or unsubstituted C₁-C₆ alkyl. In certain embodiments, R⁵ is unsubstituted C₁-C₆ alkyl.

In certain embodiments, R⁶ is substituted or unsubstituted alkyl, or substituted or unsubstituted acyl. In certain embodiments, R⁶ is substituted or unsubstituted alkyl. In certain embodiments, R⁶ is substituted or unsubstituted acyl. In certain embodiments, R⁶ is

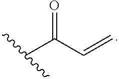 [SMILES: *C(=O)C=C]

In certain embodiments, R⁶ is

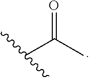 [SMILES: *C(C)=O]

In certain embodiments, each instance of R^{a}, R^{b}, and R^{c} is independently hydrogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl.

Formula (I) includes the substituent X¹. In certain embodiments, X¹ is —O—. In certain embodiments, X¹ is —N(R^{b})—. In certain embodiments, X¹ is —S—. In certain embodiments, X¹ is —C(═O)—. In certain embodiments, X¹ is —C(═O)N(R^{b})—. In certain embodiments, X¹ is —N(R^{b})C(═O)—. In certain embodiments, X¹ is —N(R^{b})C(═O)—. In certain embodiments, X¹ is —N(H)C(═O)—.

Formula (I) includes the substituents X² and X³. In certain embodiments, X² and X³ are each independently —O—, —N(R^{c})—, or —S—. In certain embodiments, X² is —O—. In certain embodiments, X² is —N(R^{c})—. In certain embodiments, X² is —S—. In certain embodiments, X³ is —O—. In certain embodiments, X³ is —N(R^{c})—. In certain embodiments, X³ is —S—.

In certain embodiments, X² is —O—, and X³ is —N(R^{c})—. In certain embodiments, X² is —O—, and X³ is —N(H)—.

In certain embodiments, X¹ is —C(═O)N(R^{b})—, X² is —O—, and X³ is —N(H)—. In certain embodiments, X¹ is —N(R^{b})C(═O)—, X² is —O—, and X³ is —N(H)—. In certain embodiments, X¹ is —C(═O)N(H)—, X² is —O—, and X³ is —N(H)—. In certain embodiments, X¹ is —N(H)C(═O)—, X² is —O—, and X³ is —N(H)—.

In certain embodiments, X¹ includes the substituent R^{b}. In certain embodiments, R^{b} is hydrogen. In certain embodiments, R^{b} is substituted or unsubstituted alkyl. In certain embodiments, R^{b} is substituted or unsubstituted alkenyl. In certain embodiments, R^{b} is substituted or unsubstituted alkynyl. In certain embodiments, R^{b} is substituted or unsubstituted acyl. In certain embodiments, R^{b} is substituted or unsubstituted carbocyclyl. In certain embodiments, R^{b} is substituted or unsubstituted heterocyclyl. In certain embodiments, R^{b} is substituted or unsubstituted aryl. In certain embodiments, R^{b} is substituted or unsubstituted heteroaryl. In certain embodiments, when attached to a nitrogen atom, R^{b} is a nitrogen protecting group.

In certain embodiments, X² or X³ includes the substituent R^{c}. In certain embodiments, R^{c} is hydrogen. In certain embodiments, R^{c} is substituted or unsubstituted alkyl. In certain embodiments, R^{c} is substituted or unsubstituted alkenyl. In certain embodiments, R^{c} is substituted or unsubstituted alkynyl. In certain embodiments, R^{c} is substituted or unsubstituted acyl. In certain embodiments, R^{c} is substituted or unsubstituted carbocyclyl. In certain embodiments, R^{c} is substituted or unsubstituted heterocyclyl. In certain embodiments, R^{c} is substituted or unsubstituted aryl. In certain embodiments, R^{c} is substituted or unsubstituted heteroaryl. In certain embodiments, when attached to a nitrogen atom, R^{c} is a nitrogen protecting group.

In certain embodiments, a compound of Formula (I) is of the Formula (II):

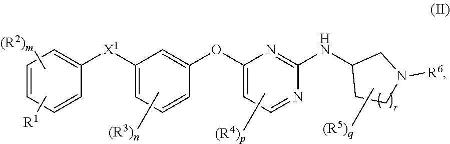

or a pharmaceutically acceptable salt, solvate, hydrate, polymorph, co-crystal, tautomer, stereoisomer, or prodrug thereof. In certain embodiments, a compound of Formula (I) is a compound of Formula (II) or a pharmaceutically acceptable salt thereof.

In certain embodiments, a compound of Formula (I) is of the Formula (III):

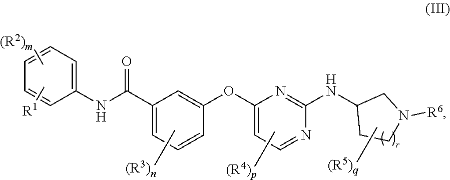

or a pharmaceutically acceptable salt, solvate, hydrate, polymorph, co-crystal, tautomer, stereoisomer, or prodrug thereof. In certain embodiments, a compound of Formula (I) is a compound of Formula (III) or a pharmaceutically acceptable salt thereof.

In certain embodiments, a compound of Formula (I) is of the Formula (IV):

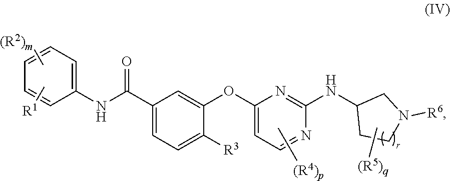

or a pharmaceutically acceptable salt, solvate, hydrate, polymorph, co-crystal, tautomer, stereoisomer, or prodrug thereof. In certain embodiments, a compound of Formula (I) is a compound of Formula (IV) or a pharmaceutically acceptable salt thereof.

In certain embodiments, a compound of Formula (I) is of the Formula (V):

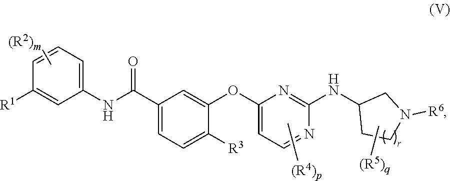

or a pharmaceutically acceptable salt, solvate, hydrate, polymorph, co-crystal, tautomer, stereoisomer, or prodrug thereof. In certain embodiments, a compound of Formula (I) is a compound of Formula (V) or a pharmaceutically acceptable salt thereof.

In certain embodiments, a compound of Formula (I) is of the Formula (VI):

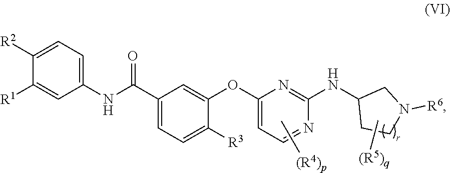

or a pharmaceutically acceptable salt, solvate, hydrate, polymorph, co-crystal, tautomer, stereoisomer, or prodrug thereof. In certain embodiments, a compound of Formula (I) is a compound of Formula (VI) or a pharmaceutically acceptable salt thereof.

In certain embodiments, a compound of Formula (I) is of the Formula (VII):

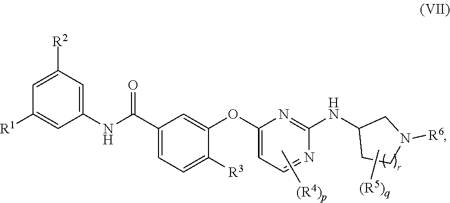

or a pharmaceutically acceptable salt, solvate, hydrate, polymorph, co-crystal, tautomer, stereoisomer, or prodrug thereof. In certain embodiments, a compound of Formula (I) is a compound of Formula (VII) or a pharmaceutically acceptable salt thereof.

In certain embodiments, a compound of Formula (I) is of the Formula (VIII):

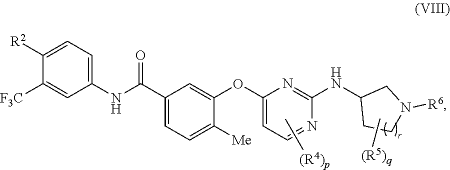

or a pharmaceutically acceptable salt, solvate, hydrate, polymorph, co-crystal, tautomer, stereoisomer, or prodrug thereof. In certain embodiments, a compound of Formula (I) is a compound of Formula (VIII) or a pharmaceutically acceptable salt thereof.

In certain embodiments, a compound of Formula (I) is of the Formula (IX):

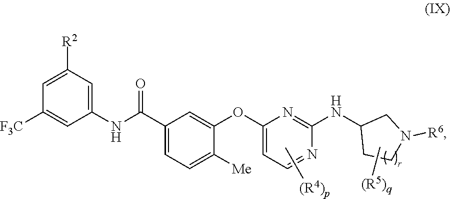

or a pharmaceutically acceptable salt, solvate, hydrate, polymorph, co-crystal, tautomer, stereoisomer, or prodrug thereof. In certain embodiments, a compound of Formula (I) is a compound of Formula (IX) or a pharmaceutically acceptable salt thereof.

In certain embodiments, a compound of Formula (I) is of the Formula (X):

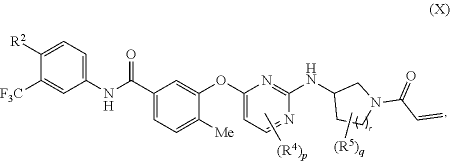

or a pharmaceutically acceptable salt, solvate, hydrate, polymorph, co-crystal, tautomer, stereoisomer, or prodrug thereof. In certain embodiments, a compound of Formula (I) is a compound of Formula (X) or a pharmaceutically acceptable salt thereof.

In certain embodiments, a compound of Formula (I) is of the Formula (XI):

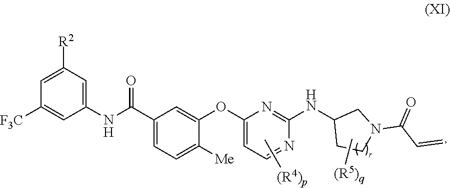

or a pharmaceutically acceptable salt, solvate, hydrate, polymorph, co-crystal, tautomer, stereoisomer, or prodrug thereof. In certain embodiments, a compound of Formula (I) is a compound of Formula (XI) or a pharmaceutically acceptable salt thereof.

In certain embodiments, a compound of Formula (I) is of the Formula (XII):

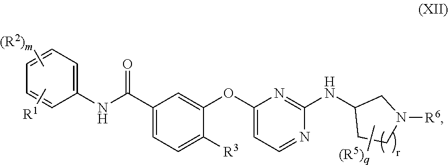

or a pharmaceutically acceptable salt, solvate, hydrate, polymorph, co-crystal, tautomer, stereoisomer, or prodrug thereof. In certain embodiments, a compound of Formula (I) is a compound of Formula (XII) or a pharmaceutically acceptable salt thereof.

In certain embodiments, a compound of Formula (I) is of the Formula (XIII):

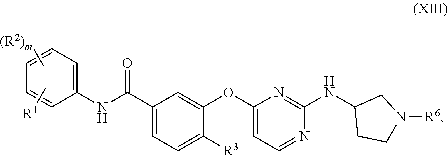

or a pharmaceutically acceptable salt, solvate, hydrate, polymorph, co-crystal, tautomer, stereoisomer, or prodrug thereof. In certain embodiments, a compound of Formula (I) is a compound of Formula (XIII) or a pharmaceutically acceptable salt thereof.

In certain embodiments a compound of Formula I is of the Formula (XIV):

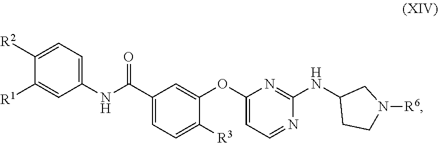

or a pharmaceutically acceptable salt, solvate, hydrate, polymorph, co-crystal, tautomer, stereoisomer, or prodrug thereof. In certain embodiments, a compound of Formula (I) is a compound of Formula (XIV) or a pharmaceutically acceptable salt thereof.

In certain embodiments, a compound of Formula (I) is of the Formula (XV):

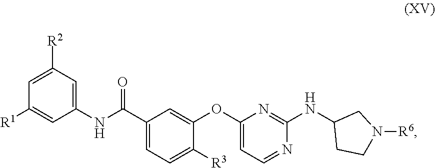

or a pharmaceutically acceptable salt, solvate, hydrate, polymorph, co-crystal, tautomer, stereoisomer, or prodrug thereof. In certain embodiments, a compound of Formula (I) is a compound of Formula (XV) or a pharmaceutically acceptable salt thereof.

In certain embodiments, a compound of Formula (I) is of the Formula (XVI):

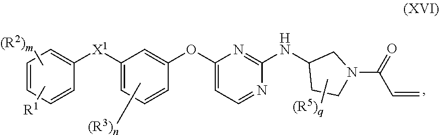

or a pharmaceutically acceptable salt, solvate, hydrate, polymorph, co-crystal, tautomer, stereoisomer, or prodrug thereof. In certain embodiments, a compound of Formula (I) is a compound of Formula (XVI) or a pharmaceutically acceptable salt thereof.

In certain embodiments, a compound of Formula (I) is of the Formula (XVII):

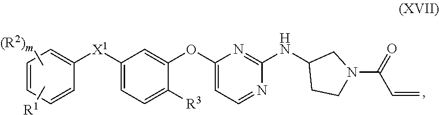

or a pharmaceutically acceptable salt, solvate, hydrate, polymorph, co-crystal, tautomer, stereoisomer, or prodrug thereof. In certain embodiments, a compound of Formula (I) is a compound of Formula (XVII) or a pharmaceutically acceptable salt thereof.

In certain embodiments, a compound of Formula (I) is of the Formula (XVIII):

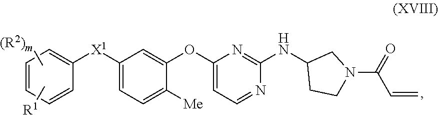

or a pharmaceutically acceptable salt, solvate, hydrate, polymorph, co-crystal, tautomer, stereoisomer, or prodrug thereof. In certain embodiments, a compound of Formula (I) is a compound of Formula (XVIII) or a pharmaceutically acceptable salt thereof.

In certain embodiments, a compound of Formula (I) is of the Formula (XIX):

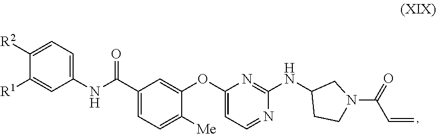

or a pharmaceutically acceptable salt, solvate, hydrate, polymorph, co-crystal, tautomer, stereoisomer, or prodrug thereof. In certain embodiments, a compound of Formula (I) is a compound of Formula (XIX) or a pharmaceutically acceptable salt thereof.

In certain embodiments, a compound of Formula (I) is of the Formula (XX):

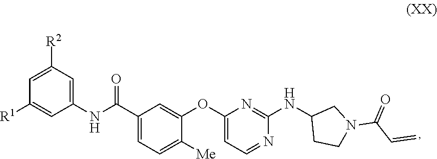

or a pharmaceutically acceptable salt, solvate, hydrate, polymorph, co-crystal, tautomer, stereoisomer, or prodrug thereof. In certain embodiments, a compound of Formula (I) is a compound of Formula (XX) or a pharmaceutically acceptable salt thereof.

In certain embodiments, a compound of Formula (I) is of the Formula (XXI):

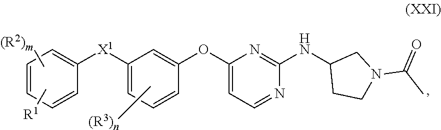

or a pharmaceutically acceptable salt, solvate, hydrate, polymorph, co-crystal, tautomer, stereoisomer, or prodrug thereof. In certain embodiments, a compound of Formula (I) is a compound of Formula (XVI) or a pharmaceutically acceptable salt thereof.

In certain embodiments, a compound of Formula (I) is of the Formula (XXII):

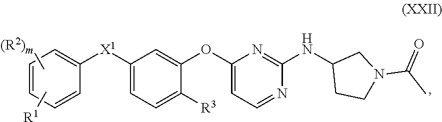

or a pharmaceutically acceptable salt, solvate, hydrate, polymorph, co-crystal, tautomer, stereoisomer, or prodrug thereof. In certain embodiments, a compound of Formula (I) is a compound of Formula (XVII) or a pharmaceutically acceptable salt thereof.

In certain embodiments, a compound of Formula (I) is of the Formula (XVIII):

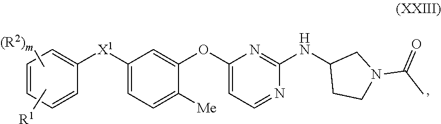

or a pharmaceutically acceptable salt, solvate, hydrate, polymorph, co-crystal, tautomer, stereoisomer, or prodrug thereof. In certain embodiments, a compound of Formula (I) is a compound of Formula (XVIII) or a pharmaceutically acceptable salt thereof.

In certain embodiments, a compound of Formula (I) is of the Formula (XXIV):

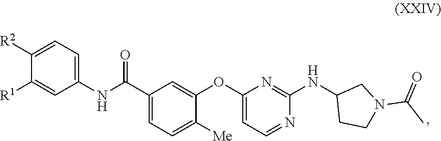

or a pharmaceutically acceptable salt, solvate, hydrate, polymorph, co-crystal, tautomer, stereoisomer, or prodrug thereof. In certain embodiments, a compound of Formula (I) is a compound of Formula (XXIV) or a pharmaceutically acceptable salt thereof.

In certain embodiments, a compound of Formula (I) is of the Formula (XV):

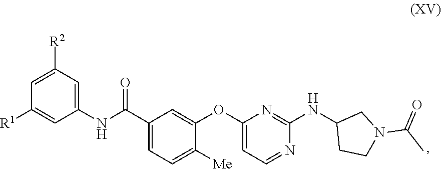

or a pharmaceutically acceptable salt, solvate, hydrate, polymorph, co-crystal, tautomer, stereoisomer, or prodrug thereof. In certain embodiments, a compound of Formula (I) is a compound of Formula (XX) or a pharmaceutically acceptable salt thereof.

In certain embodiments, the compound of Formula (I) is of the formula:

| Compound | Formula |
|---|---|
| (I-1) | 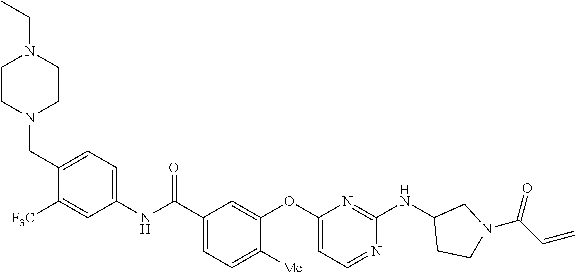 [SMILES: C=CC(=O)N1CCC(Nc2nccc(Oc3cc(C(=O)Nc4ccc(CN5CCN(CC)CC5)c(C(F)(F)F)c4)ccc3C)n2)C1] |
| (I-2) | 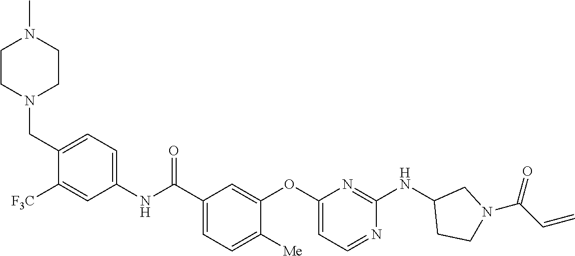 [SMILES: C=CC(=O)N1CCC(Nc2nccc(Oc3cc(C(=O)Nc4ccc(CN5CCN(C)CC5)c(C(F)(F)F)c4)ccc3C)n2)C1] |
| (I-3) | 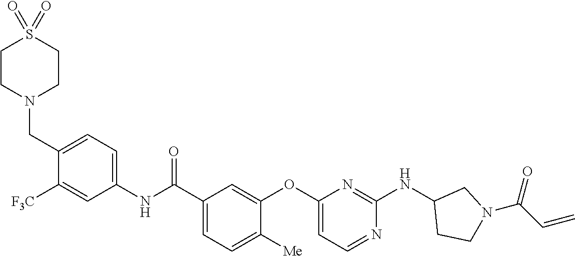 [SMILES: C=CC(=O)N1CCC(Nc2nccc(Oc3cc(C(=O)Nc4ccc(CN5CCS(=O)(=O)CC5)c(C(F)(F)F)c4)ccc3C)n2)C1] |
| (I-4) | 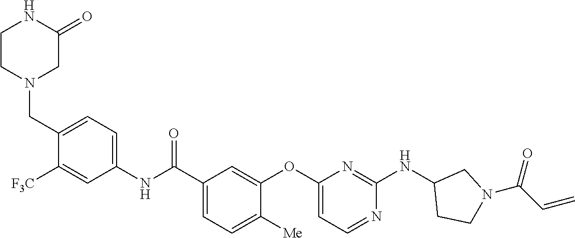 [SMILES: C=CC(=O)N1CCC(Nc2nccc(Oc3cc(C(=O)Nc4ccc(CN5CCNC(=O)C5)c(C(F)(F)F)c4)ccc3C)n2)C1] |
| (I-5) | 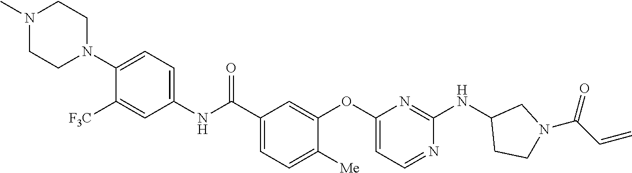 [SMILES: C=CC(=O)N1CCC(Nc2nccc(Oc3cc(C(=O)Nc4ccc(N5CCN(C)CC5)c(C(F)(F)F)c4)ccc3C)n2)C1] |
| (I-6) | 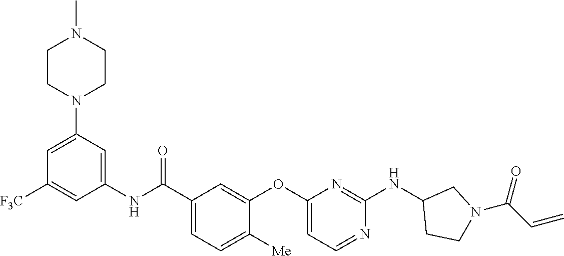 [SMILES: C=CC(=O)N1CCC(Nc2nccc(Oc3cc(C(=O)Nc4cc(N5CCN(C)CC5)cc(C(F)(F)F)c4)ccc3C)n2)C1] |
| (I-7) | 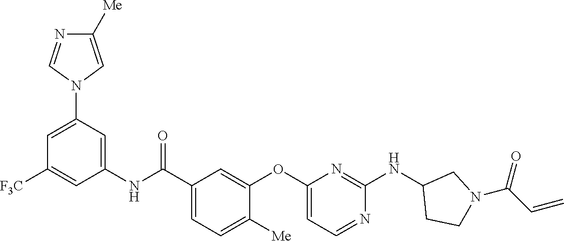 [SMILES: C=CC(=O)N1CCC(Nc2nccc(Oc3cc(C(=O)Nc4cc(-n5cnc(C)c5)cc(C(F)(F)F)c4)ccc3C)n2)C1] |
| (I-8) | 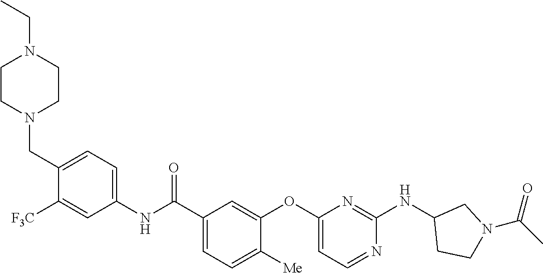 [SMILES: CCN1CCN(Cc2ccc(NC(=O)c3ccc(C)c(Oc4ccnc(NC5CCN(C(C)=O)C5)n4)c3)cc2C(F)(F)F)CC1] |
| (I-9) | 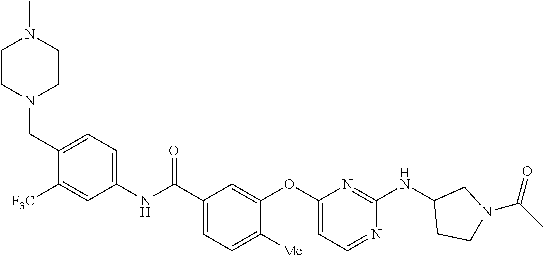 [SMILES: CC(=O)N1CCC(Nc2nccc(Oc3cc(C(=O)Nc4ccc(CN5CCN(C)CC5)c(C(F)(F)F)c4)ccc3C)n2)C1] |
| (I-10) | 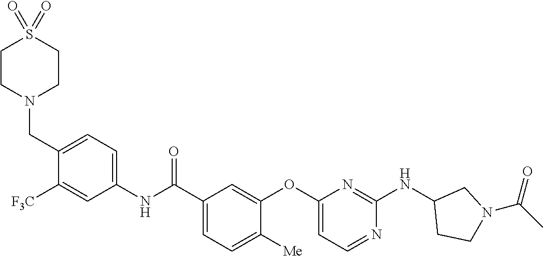 [SMILES: CC(=O)N1CCC(Nc2nccc(Oc3cc(C(=O)Nc4ccc(CN5CCS(=O)(=O)CC5)c(C(F)(F)F)c4)ccc3C)n2)C1] |
| (I-11) | 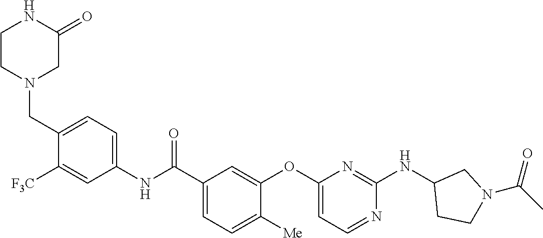 [SMILES: CC(=O)N1CCC(Nc2nccc(Oc3cc(C(=O)Nc4ccc(CN5CCNC(=O)C5)c(C(F)(F)F)c4)ccc3C)n2)C1] |
| (I-12) | 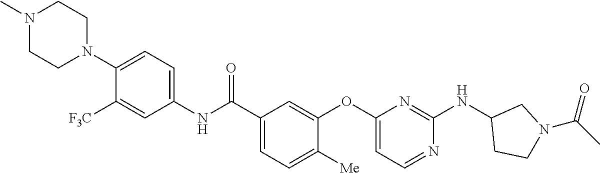 [SMILES: CC(=O)N1CCC(Nc2nccc(Oc3cc(C(=O)Nc4ccc(N5CCN(C)CC5)c(C(F)(F)F)c4)ccc3C)n2)C1] |
| (I-13) | 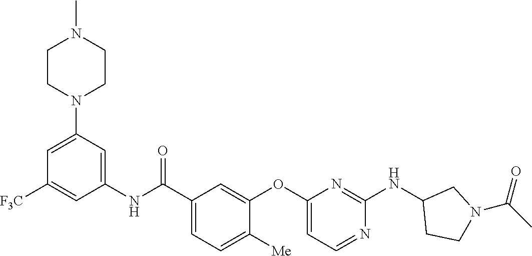 [SMILES: CC(=O)N1CCC(Nc2nccc(Oc3cc(C(=O)Nc4cc(N5CCN(C)CC5)cc(C(F)(F)F)c4)ccc3C)n2)C1] |
| (I-14) | 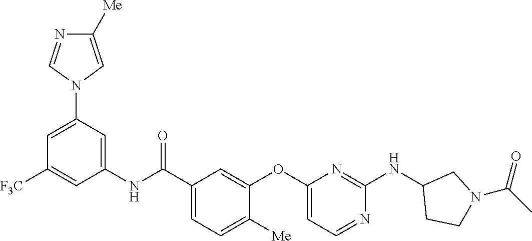 [SMILES: CC(=O)N1CCC(Nc2nccc(Oc3cc(C(=O)Nc4cc(-n5cnc(C)c5)cc(C(F)(F)F)c4)ccc3C)n2)C1] |

or a pharmaceutically acceptable salt, solvate, hydrate, polymorph, co-crystal, tautomer, stereoisomer, isotopically labeled derivative, or prodrug thereof.

In certain embodiments, the compound of Formula (I) is of the Formula S-(I-1):

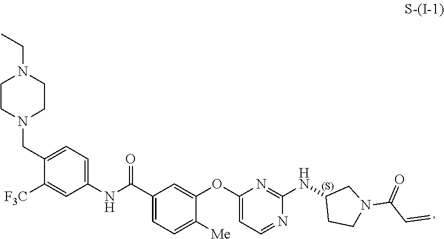

or a pharmaceutically acceptable salt, solvate, hydrate, polymorph, co-crystal, tautomer, stereoisomer, isotopically labeled derivative, or prodrug thereof. In certain embodiments, a compound of Formula (I) is a compound of Formula S-(I-1) or a pharmaceutically acceptable salt thereof.

In certain embodiments, the compound of Formula (I) is of the Formula R-(I-1):

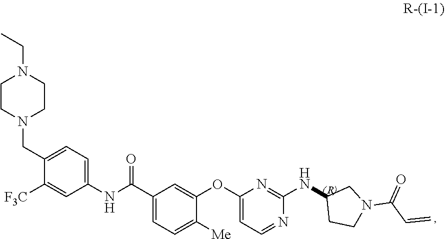

or a pharmaceutically acceptable salt, solvate, hydrate, polymorph, co-crystal, tautomer, stereoisomer, isotopically labeled derivative, or prodrug thereof. In certain embodiments, a compound of Formula (I) is a compound of Formula R-(I-1) or a pharmaceutically acceptable salt thereof.

In certain embodiments, a compound described herein is a compound of Formula (I), or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I), or a pharmaceutically acceptable salt thereof.

### Pharmaceutical Compositions, Kits, and Administration

The present disclosure also provides pharmaceutical compositions comprising a compound of Formula (I) as described herein, or a pharmaceutically acceptable salt thereof, and optionally a pharmaceutically acceptable excipient. In certain embodiments, a compound described herein is a compound of Formula (I), or a pharmaceutically acceptable salt thereof, and a pharmaceutically acceptable excipient.

In certain embodiments, the compound described herein is provided in the pharmaceutical composition in an amount effective for inhibiting the activity (e.g., aberrant activity, such as increased activity) of a protein kinase (e.g., MAP2K (e.g., MKK7). In certain embodiments, the compound described herein is provided in the pharmaceutical composition in a therapeutically effective amount. In certain embodiments, the compound described herein is provided in the pharmaceutical composition in a prophylactically effective amount.

In certain embodiments, the compound described herein is provided in the pharmaceutical composition in an amount effective for treating an infectious disease (e.g., a viral infection (e.g., infections caused by viral human hepatitis (e.g., hepatitis B), a flaviviridae family virus (e.g., a flavivirus (e.g., Zika virus)))) in a subject in need thereof. In certain embodiments, the compound described herein is provided in the pharmaceutical composition in an amount effective for preventing an infectious disease (e.g., a viral infection (e.g., infections caused by viral human hepatitis (e.g., hepatitis B), a flaviviridae family virus (e.g., a flavivirus (e.g., Zika virus)))) in a subject in need thereof.

In certain embodiments, the compound described herein is provided in the pharmaceutical composition in an amount effective for treating a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof. In certain embodiments, the compound described herein is provided in the pharmaceutical composition in an amount effective for preventing a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof.

In certain embodiments, the compound described herein is provided in the pharmaceutical composition in an amount effective for reducing the risk of developing a disease (e.g., an inflammatory disease, neurological disease (e.g., Alzheimer's disease), infectious disease (e.g., a viral infection (e.g., an infection caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof.

In certain embodiments, the compound described herein is provided in the pharmaceutical composition in an amount effective for treating an inflammatory disease in a subject in need thereof. In certain embodiments, the compound described herein is provided in the pharmaceutical composition in an amount effective for preventing an inflammatory disease in a subject in need thereof.

In certain embodiments, the compound described herein is provided in the pharmaceutical composition in an amount effective for treating a neurological disease (e.g., Alzheimer's disease) in a subject in need thereof. In certain embodiments, the compound described herein is provided in the pharmaceutical composition in an amount effective for preventing a neurological disease (e.g., Alzheimer's disease) in a subject in need thereof.

In certain embodiments, the compound described herein is provided in the pharmaceutical composition in an amount effective for inhibiting the activity (e.g., aberrant activity, such as increased activity) of a protein kinase (e.g., MAP2K (e.g., MKK7) in a subject or cell. In certain embodiments, the compound described herein is provided in the pharmaceutical composition in an amount effective for inhibiting the activity (e.g., aberrant activity, such as increased activity) of a Mitogen-activated protein kinase kinase (MAP2K) (e.g., MKK7).

In certain embodiments, the compound described herein is provided in the pharmaceutical composition in an amount effective for inhibiting the activity of a protein kinase (e.g., MAP2K (e.g., MKK7)) by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of a MAP2K family kinase (e.g., MKK7) by not more than 10%, not more than 20%, not more than 30%, not more than 40%, not more than 50%, not more than 60%, not more than 70%, not more than 80%, not more than 90%, not more than 95%, or not more than 98%. In certain embodiments the compound described herein is provided in the pharmaceutical composition in an amount effective for inhibiting the activity of a MAP2K family kinase (e.g., MKK7) by a range between a percentage described in this paragraph and another percentage described in this paragraph, inclusive.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragées, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample or cell, any two doses of the multiple doses include different or substantially the same amounts of a compound described herein. In certain embodiments, when multiple doses are administered to a subject or applied to a biological sample or cell, the frequency of administering the multiple doses to the subject or applying the multiple doses to the biological sample or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample or cell is one dose per day. In certain embodiments, the frequency of administering the multiple doses to the subject or applying the multiple doses to the biological sample or cell is two doses per day. In certain embodiments, the frequency of administering the multiple doses to the subject or applying the multiple doses to the biological sample or cell is three doses per day. In certain embodiments, when multiple doses are administered to a subject or applied to a biological sample or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, biological sample, or cell. In certain embodiments, the duration between the first dose and last dose of the multiple doses is three months, six months, or one year. In certain embodiments, the duration between the first dose and last dose of the multiple doses is the lifetime of the subject, biological sample, or cell. In certain embodiments, a dose (e.g., a single dose, or any dose of multiple doses) described herein includes independently between 0.1 pg and 1 pg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-inflammatory agent. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof. In certain embodiments, the kits are useful for preventing a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof. In certain embodiments, the kits are useful for reducing the risk of developing a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof. In certain embodiments, the kits are useful for inhibiting the activity (e.g., aberrant activity, such as increased activity) of a protein kinase (e.g., MAP2K (e.g., MKK7)) in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof. In certain embodiments, the kits and instructions provide for preventing a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof. In certain embodiments, the kits and instructions provide for reducing the risk of developing a disease (e.g., an inflammatory disease, neurological disease (e.g., Alzheimer's disease), infectious disease (e.g., a viral infection (e.g., infections caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)) in a subject in need thereof. In certain embodiments, the kits and instructions provide for inhibiting the activity (e.g., aberrant activity, such as increased activity) of a protein kinase (e.g., MAP2K (e.g., MKK7)) in a subject or cell. A kit described herein may include one or more additional pharmaceutical agents described herein as a separate composition.

### Methods of Treatment and Uses

The present disclosure provides methods of using the compounds described herein, e.g., as therapeutics, e.g., in the treatment and/or prevention of diseases associated with the overexpression and/or aberrant activity of a protein kinase (e.g., MAP2K (e.g., MKK7)), and as biological probes to study the inhibition of the activity of a protein kinase (e.g., MAP2K (e.g., MKK7)). In certain embodiments, the compound covalently binds a protein kinase (e.g., MAP2K (e.g., MKK7)). In certain embodiments, the diseases treated and/or prevented include, but are not limited to inflammatory diseases. In certain embodiments, the diseases treated and/or prevented include, but are not limited to neurological diseases (e.g., Alzheimer's disease). In certain embodiments, the diseases treated and/or prevented include, but are not limited to infectious diseases (e.g., viral infections (e.g., infections caused by viral human hepatitis (e.g., hepatitis B), a flaviviridae family virus (e.g., a flavivirus (e.g., Zika virus)))). In certain embodiments, the diseases treated and/or prevented include, but are not limited to proliferative diseases (e.g., cancer (e.g., lung cancer, prostate cancer, skin cancer, thyroid cancer, pancreatic cancer, colorectal cancer, liver cancer, leukemia, or lymphoma)). In certain embodiments, the cancer is associated with the overexpression and/or aberrant activity of a protein kinase (e.g., MAP2K (e.g., MKK7)).

The compounds described herein may be useful in treating and/or preventing diseases (e.g., inflammatory diseases, neurological diseases (e.g., Alzheimer's disease), infectious diseases (e.g., viral infections (e.g., infections caused by viral human hepatitis (e.g., hepatitis B), a flaviviridae family virus (e.g., a flavivirus (e.g., Zika virus)))), and proliferative diseases (e.g., cancer (e.g., lung cancer, prostate cancer, skin cancer, thyroid cancer, pancreatic cancer, colorectal cancer, liver cancer, leukemia, or lymphoma)) or diseases associated with the activity of a MAP2K (e.g., MKK7) in a subject, or inhibiting the activity of a MAP2K in a subject, biological sample, or cell. In certain embodiments, a compound described herein is a compound of Formula (I), or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I), or a pharmaceutically acceptable salt thereof.

Certain compounds described herein bind, covalently modify, and/or inhibit a protein kinase (e.g., MAP2K (e.g., MKK7)). In certain embodiments, the compounds described herein irreversibly inhibit a protein kinase. In certain embodiments, the compounds described herein reversibly inhibit a protein kinase. In certain embodiments, the protein kinase is a member of the mitogen-activated protein kinase kinase (MAP2K) subfamily involved in the c-Jun N-terminal kinase (JNK) signaling pathway. In certain embodiments, the protein kinase is mitogen-activated protein kinase kinase (i.e., MKK7, MEK7, MAP2K7). In certain embodiments, the compounds described herein are Type II MAP2K inhibitors. In certain embodiments, the compounds described herein are Type II MKK7 inhibitors.

In certain embodiments, the compound described herein modulates the activity of a protein kinase (e.g., MAP2K (e.g., MKK7)). In certain embodiments, the compounds described herein inhibit the activity of a protein kinase (e.g., MAP2K (e.g., MKK7)). In certain embodiments, the compounds described herein reversibly inhibit the activity of a protein kinase (e.g., MAP2K (e.g., MKK7)). In certain embodiments, the compounds described herein irreversibly inhibit the activity of a protein kinase (e.g., MAP2K (e.g., MKK7)).

The binding affinity of a compound described herein to a protein kinase (e.g., MAP2K (e.g., MKK7)) may be measured by the dissociation constant (K_{d}) value of an adduct of the compound and the protein kinase (e.g., MAP2K (e.g., MKK7)) using methods known in the art (e.g., isothermal titration calorimetry (ITC)). In certain embodiments, the K_{d} value of the adduct is not more than about 100 μM, not more than about 10 μM not more than about 1 μM not more than about 100 nM, not more than about 10 nM, or not more than about 1 nM.

In certain embodiments, the activity of a protein kinase (e.g., MAP2K (e.g., MKK7)) is inhibited by a compound described herein. The inhibition of the activity of a protein kinase (e.g., MAP2K (e.g., MKK7)) by a compound described herein may be measured by determining the half maximal inhibitory concentration (IC₅₀) of the compound when the compound, or a pharmaceutical composition thereof, is contacted with the protein kinase (e.g., MAP2K (e.g., MKK7)). The IC₅₀ values may be obtained using methods known in the art (e.g., by a competition binding assay). In certain embodiments, the IC₅₀ value of a compound described herein is not more than about 1 mM, not more than about 100 μM, not more than about 10 μM not more than about 1 μM not more than about 100 nM, not more than about 10 nM, or not more than about 1 nM.

The compounds described herein may selectively modulate the activity of a protein kinase (e.g., MAP2K (e.g., MKK7)). In certain embodiments, the compounds selectively increase the activity of a protein kinase (e.g., MAP2K (e.g., MKK7)). In certain embodiments, the compounds selectively inhibit the activity of a protein kinase (e.g., MAP2K (e.g., MKK7)). In certain embodiments, the compounds inhibit the activity of two or more protein kinases (e.g., MAP2K (e.g., MKK7)) to the same extent.

The selectivity of a compound described herein in inhibiting the activity of a first protein kinase (e.g., MAP2K (e.g., MKK7)) over a second protein kinase may be measured by the quotient of the IC₅₀ value of the compound in inhibiting the activity of the second protein kinase (e.g., MAP2K (e.g., MKK7)) over the IC₅₀ value of the compound in inhibiting the activity of the first protein kinase. The selectivity of a compound described herein in modulating the activity of a first protein kinase (e.g., MAP2K (e.g., MKK7)) over a second protein kinase may also be measured by the quotient of the K_{d} value of an adduct of the compound and the second protein kinase over the K_{d} value of an adduct of the compound and the first protein kinase (e.g., MAP2K (e.g., MKK7)). In certain embodiments, the selectivity is at least about 1-fold, at least about 3-fold, at least about 10-fold, at least about 30-fold, at least about 100-fold, at least about 300-fold, at least about 1,000-fold, at least about 3,000-fold, at least about 10,000-fold, at least about 30,000-fold, or at least about 100,000-fold.

The present disclosure provides methods of modulating (e.g., inhibiting or increasing) the activity (e.g., aberrant activity, such as increased or decreased activity) of a protein kinase (e.g., MAP2K (e.g., MKK7)). The present disclosure provides methods of modulating (e.g., inhibiting or increasing) the activity (e.g., aberrant activity, such as increased or decreased activity) of a MAP2K (e.g., MKK7) in a subject, biological sample, or cell. The present disclosure also provides methods for the treatment of a wide range of diseases, such as diseases associated with the aberrant activity (e.g., increased activity) of a protein kinase (e.g., MAP2K (e.g., MKK7)), in a subject in need thereof. The present disclosure also provides methods for the treatment and/or prevention of an inflammatory disease, neurological disease (e.g., Alzheimer's disease), infectious disease (e.g., a viral infection (e.g., an infection caused by viral human hepatitis (e.g., hepatitis B), a flaviviridae family virus (e.g., a flavivirus (e.g., Zika virus)))), or a proliferative disease (e.g., cancer (e.g., lung cancer, prostate cancer, skin cancer, thyroid cancer, pancreatic cancer, colorectal cancer, liver cancer, leukemia, or lymphoma)).

The present disclosure also provides a compound of Formula (I), or a pharmaceutically acceptable salt, co-crystal, tautomer, stereoisomer, solvate, hydrate, polymorph, isotopically enriched derivative, or prodrug, or composition thereof, for use in the treatment of diseases, such as inflammatory diseases, neurological diseases (e.g., Alzheimer's disease), infectious diseases (e.g., viral infections (e.g., infections caused by viral human hepatitis (e.g., hepatitis B), a flaviviridae family virus (e.g., a flavivirus (e.g., Zika virus)))), and proliferative diseases (e.g., cancer (e.g., lung cancer, prostate cancer, skin cancer, thyroid cancer, pancreatic cancer, colorectal cancer, liver cancer, leukemia, or lymphoma)), in a subject in need thereof.

The present disclosure also provides uses of a compound of Formula (I), or a pharmaceutically acceptable salt, co-crystal, tautomer, stereoisomer, solvate, hydrate, polymorph, isotopically enriched derivative, or prodrug, or composition thereof, in the manufacture of a medicament for the treatment of diseases, such as inflammatory diseases, neurological diseases (e.g., Alzheimer's disease), infectious diseases (e.g., viral infections (e.g., infections caused by viral human hepatitis (e.g., hepatitis B), a flaviviridae family virus (e.g., a flavivirus (e.g., Zika virus)))), and proliferative diseases (e.g., cancer (e.g., lung cancer, prostate cancer, skin cancer, thyroid cancer, pancreatic cancer, colorectal cancer, liver cancer, leukemia, or lymphoma)), in a subject in need thereof.

In certain embodiments, provided are methods of decreasing the activity of a protein kinase (e.g., MAP2K (e.g., MKK7)) in a subject, biological sample, or cell by a method described herein by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a protein kinase (e.g., MAP2K (e.g., MKK7)) in a subject, biological sample, or cell is decreased by a method described herein by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a protein kinase (e.g., MAP2K (e.g., MKK7)) in a subject, biological sample, or cell is selectively inhibited by the method. In some embodiments, the activity of a protein kinase (e.g., MAP2K (e.g., MKK7)) in a subject, biological sample, or cell is selectively decreased by the method.

Without wishing to be bound by any particular theory, the compounds described herein are able to bind (e.g., covalently modify) the protein kinase (e.g., MAP2K (e.g., MKK7)) being inhibited. In certain embodiments, a compound described herein is able to bind (e.g., covalently modify) the protein kinase (e.g., MAP2K (e.g., MKK7)).

In another aspect, the present disclosure provides methods of inhibiting the activity of a protein kinase (e.g., MAP2K (e.g., MKK7)) in a subject, the methods comprising administering to the subject an effective amount (e.g., a therapeutically effective amount) of a compound, or pharmaceutical composition thereof, as described herein. In another aspect, the present disclosure provides methods of inhibiting the activity of a protein kinase MAP2K (e.g., MKK7)) in a biological sample or cell, the methods comprising contacting the biological sample or cell with an effective amount of a compound, or pharmaceutical composition thereof, as described herein.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs).

In certain embodiments, the biological sample being contacted with the compound or composition is breast tissue, bone marrow, lymph node, lymph tissue, spleen, or blood. In certain embodiments, the biological sample being contacted with the compound or composition is a tumor or cancerous tissue. In certain embodiments, the biological sample being contacted with the compound or composition is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

In certain embodiments, the biological sample or cell being contacted with the compound or composition is present in vitro. In certain embodiments, the biological sample or cell being contacted with the compound or composition is present in vivo. In certain embodiments, the biological sample or cell being contacted with the compound or composition is present ex vivo.

The disease (e.g., proliferative disease) to be treated or prevented using the compounds described herein may be associated with increased activity of a protein kinase, such as MAP2K (e.g., MKK7)). The disease (e.g., inflammatory disease, neurological disease (e.g., Alzheimer's disease), infectious disease (e.g., viral infection (e.g., infections caused by viral human hepatitis (e.g., hepatitis B), a flaviviridae family virus (e.g., a flavivirus (e.g., Zika virus)))), or proliferative disease (e.g., cancer (e.g., lung cancer, prostate cancer, skin cancer, thyroid cancer, pancreatic cancer, colorectal cancer, liver cancer, leukemia, or lymphoma)).) to be treated or prevented using the compounds described herein may be associated with the overexpression of a protein kinase (e.g., MAP2K (e.g., MKK7)).

In certain embodiments, the disease (e.g., inflammatory disease, neurological disease (e.g., Alzheimer's disease), infectious disease (e.g., viral infections (e.g., infections caused by viral human hepatitis (e.g., hepatitis B), a flaviviridae family virus (e.g., a flavivirus (e.g., Zika virus)))), or proliferative disease (e.g., cancer (e.g., lung cancer, prostate cancer, skin cancer, thyroid cancer, pancreatic cancer, colorectal cancer, liver cancer, leukemia, or lymphoma)) to be treated or prevented using the compounds described herein may be associated with the overexpression of a MAP2K (e.g., MKK7). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a protein kinase (e.g., MAP2K (e.g., MKK7)) and be useful in treating and/or preventing diseases (e.g., inflammatory diseases, neurological diseases (e.g., Alzheimer's disease), infectious diseases (e.g., viral infections (e.g., infections caused by viral human hepatitis (e.g., hepatitis B), a flaviviridae family virus (e.g., a flavivirus (e.g., Zika virus)))), and proliferative diseases (e.g., cancer (e.g., lung cancer, prostate cancer, skin cancer, thyroid cancer, pancreatic cancer, colorectal cancer, liver cancer, leukemia, or lymphoma)). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a protein kinase (e.g., MAP2K (e.g., MKK7)) and be useful in treating and/or preventing diseases (e.g., inflammatory diseases, neurological diseases (e.g., Alzheimer's disease), infectious diseases (e.g., viral infections (e.g., infections caused by viral human hepatitis (e.g., hepatitis B), a flaviviridae family virus (e.g., a flavivirus (e.g., Zika virus)))), and proliferative diseases (e.g., cancer (e.g., lung cancer, prostate cancer, skin cancer, thyroid cancer, pancreatic cancer, colorectal cancer, liver cancer, leukemia, or lymphoma)).

Another aspect of the disclosure relates to methods of inhibiting the activity of a protein kinase (e.g., MAP2K (e.g., MKK7)) in a subject, biological sample, or cell.

In certain embodiments, the methods described herein include administering to a subject or contacting a biological sample with an effective amount (e.g., a therapeutically effective amount) of a compound described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the methods described herein include administering to a subject or contacting a biological sample with an effective amount (e.g., a therapeutically effective amount) of a compound described herein, or a pharmaceutically acceptable salt thereof, or a pharmaceutical composition thereof. In certain embodiments, the compound is contacted with a biological sample. In certain embodiments, the compound is administered to a subject. In certain embodiments, the compound is administered in combination with one or more additional pharmaceutical agents described herein.

In some embodiments, the activity of a protein kinase (e.g., MAP2K (e.g., MKK7)) is non-selectively inhibited by the compounds or pharmaceutical compositions described herein. In some embodiments, the activity of the protein kinase (e.g., MAP2K (e.g., MKK7)) being inhibited is selectively inhibited by the compounds or pharmaceutical compositions described herein, compared to the activity of a different protein (e.g., a different protein kinase). In certain embodiments, the activity of a MAP2K (e.g., MKK7) is selectively inhibited by a compound or pharmaceutical composition described herein, compared to the activity of a different protein. In certain embodiments, the activity of MKK7 is selectively inhibited by a compound or pharmaceutical composition described herein, compared to the activity of another MAP2K.

In certain embodiments, the activity of JNK1 is selectively inhibited by a compound or pharmaceutical composition described herein, compared to the activity of another JNK. In certain embodiments, the activity of JNK2 is selectively inhibited by a compound or pharmaceutical composition described herein, compared to the activity of another JNK.

The selectivity of a compound or pharmaceutical composition described herein in inhibiting the activity of a protein kinase (e.g., MAP2K (e.g., MKK7)) over a different protein (e.g., a different protein kinase) may be measured by the quotient of the IC₅₀ value of the compound or pharmaceutical composition in inhibiting the activity of the different protein over the IC₅₀ value of the compound or pharmaceutical composition in inhibiting the activity of the protein kinase. The selectivity of a compound or pharmaceutical composition described herein for a protein kinase over a different protein may also be measured by the quotient of the K_{d} value of an adduct of the compound or pharmaceutical composition and the different protein over the K_{d} value of an adduct of the compound or pharmaceutical composition and the protein kinase. In certain embodiments, the selectivity is at least 2-fold, at least 3-fold, at least 5-fold, at least 10-fold, at least 30-fold, at least 100-fold, at least 300-fold, at least 1,000-fold, at least 3,000-fold, at least 10,000-fold, at least 30,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

### Example 1. KINOMEscan Ambit Data for Compound S-(I-1)

The activity of the compounds was confirmed using Compound S-(I-1) in the KINOMEscan screening platform. The results are shown in Table 1.

TABLE 1
KINOMEscan Ambit Data for
Compound S-(I-1).

| Kinase | Score |
|---|---|
| AAK1 | 82 |
| ABL1(E255K)- | 1.6 |
| phosphorylated | |
| ABL1(F317I)- | 1.3 |
| nonphosphorylated | |
| ABL1(F317I)- | 7.6 |
| phosphorylated | |
| ABL1(F317L)- | 1.6 |
| nonphosphorylated | |
| ABL1(F317L)- | 3.3 |
| phosphorylated | |
| ABL1(H396P)- | 0 |
| nonphosphorylated | |
| ABL1(H396P)- | 2.3 |
| phosphorylated | |
| ABL1(M351T)- | 2.9 |
| phosphorylated | |
| ABL1(Q252H)- | 0.8 |
| nonphosphorylated | |
| ABL1(Q252H)- | 2.4 |
| phosphorylated | |
| ABL1(T315I)- | 5 |
| nonphosphorylated | |
| ABL1(T315I)- | 62 |
| phosphorylated | |
| ABL1(Y253F)- | 1 |
| phosphorylated | |
| ABL1- | 1 |
| nonphosphorylated | |
| ABL1-phosphorylated | 2 |
| ABL2 | 5 |
| ACVR1 | 100 |
| ACVR1B | 92 |
| ACVR2A | 94 |
| ACVR2B | 93 |
| ACVRL1 | 85 |
| ADCK3 | 100 |
| ADCK4 | 96 |
| AKT1 | 100 |
| AKT2 | 98 |
| AKT3 | 94 |
| ALK | 82 |
| ALK(C1156Y) | 100 |
| ALK(L1196M) | 78 |
| AMPK-alpha1 | 92 |
| AMPK-alpha2 | 86 |
| ANKK1 | 100 |
| ARK5 | 83 |
| ASK1 | 94 |
| ASK2 | 87 |
| AURKA | 92 |
| AURKB | 98 |
| AURKC | 100 |
| AXL | 78 |
| BIKE | 96 |
| BLK | 0.5 |
| BMPR1A | 96 |
| BMPR1B | 93 |
| BMPR2 | 100 |
| BMX | 54 |
| BRAF | 82 |
| BRAF(V600E) | 41 |
| BRK | 100 |
| BRSK1 | 100 |
| BRSK2 | 100 |
| BTK | 98 |
| BUB1 | 88 |
| CAMK1 | 29 |
| CAMK1D | 28 |
| CAMK1G | 60 |
| CAMK2A | 95 |
| CAMK2B | 100 |
| CAMK2D | 88 |
| CAMK2G | 89 |
| CAMK4 | 73 |
| CAMKK1 | 81 |
| CAMKK2 | 69 |
| CASK | 81 |
| CDC2L1 | 36 |
| CDC2L2 | 37 |
| CDC2L5 | 65 |
| CDK11 | 0 |
| CDK2 | 77 |
| CDK3 | 56 |
| CDK4-cyclinD1 | 95 |
| CDK4-cyclinD3 | 100 |
| CDK5 | 72 |
| CDK7 | 49 |
| CDK8 | 2.8 |
| CDK9 | 72 |
| CDKL1 | 77 |
| CDKL2 | 19 |
| CDKL3 | 24 |
| CDKL5 | 74 |
| CHEK1 | 100 |
| CHEK2 | 94 |
| CIT | 61 |
| CLK1 | 85 |
| CLK2 | 98 |
| CLK3 | 96 |
| CLK4 | 82 |
| CSF1R | 2.8 |
| CSF1R-autoinhibited | 95 |
| CSK | 58 |
| CSNK1A1 | 88 |
| CSNK1A1L | 96 |
| CSNK1D | 100 |
| CSNK1E | 90 |
| CSNK1G1 | 91 |
| CSNK1G2 | 97 |
| CSNK1G3 | 92 |
| CSNK2A1 | 100 |
| CSNK2A2 | 93 |
| CTK | 74 |
| DAPK1 | 90 |
| DAPK2 | 99 |
| DAPK3 | 100 |
| DCAMKL1 | 99 |
| DCAMKL2 | 75 |
| DCAMKL3 | 100 |
| DDR1 | 0.4 |
| DDR2 | 12 |
| DLK | 90 |
| DMPK | 98 |
| DMPK2 | 96 |
| DRAK1 | 88 |
| DRAK2 | 98 |
| DYRK1A | 100 |
| DYRK1B | 91 |
| DYRK2 | 85 |
| EGFR | 68 |
| EGFR(E746-A750del) | 66 |
| EGFR(G719C) | 59 |
| EGFR(G719S) | 81 |
| EGFR(L747-E749del, | 52 |
| A750P) | |
| EGFR(L747-S752del, | 38 |
| P753S) | |
| EGFR(L747- | 43 |
| T751del, Sins) | |
| EGFR(L858R) | 84 |
| EGFR(L858R, T790M) | 94 |
| EGFR(L861Q) | 66 |
| EGFR(S752-I759del) | 90 |
| EGFR(T790M) | 90 |
| EIF2AK1 | 91 |
| EPHA1 | 87 |
| EPHA2 | 70 |
| EPHA3 | 45 |
| EPHA4 | 85 |
| EPHA5 | 87 |
| EPHA6 | 98 |
| EPHA7 | 97 |
| EPHA8 | 12 |
| EPHB1 | 100 |
| EPHB2 | 91 |
| EPHB3 | 99 |
| EPHB4 | 95 |
| EPHB6 | 76 |
| ERBB2 | 95 |
| ERBB3 | 98 |
| ERBB4 | 98 |
| ERK1 | 100 |
| ERK2 | 87 |
| ERK3 | 99 |
| ERK4 | 91 |
| ERK5 | 87 |
| ERK8 | 97 |
| ERN1 | 96 |
| FAK | 99 |
| FER | 40 |
| FES | 5.3 |
| FGFR1 | 55 |
| FGFR2 | 77 |
| FGFR3 | 93 |
| FGFR3(G697C) | 84 |
| FGFR4 | 51 |
| FGR | 14 |
| FLT1 | 36 |
| FLT3 | 7 |
| FLT3(D835H) | 52 |
| FLT3(D835Y) | 48 |
| FLT3(ITD) | 24 |
| FLT3(K663Q) | 4.2 |
| FLT3(N841I) | 20 |
| FLT3(R834Q) | 59 |
| FLT3-autoinhibited | 79 |
| FLT4 | 62 |
| FRK | 11 |
| FYN | 39 |
| GAK | 52 |
| GCN2(Kin.Dom.2, | 99 |
| S808G) | |
| GRK1 | 79 |
| GRK4 | 92 |
| GRK7 | 96 |
| GSK3A | 72 |
| GSK3B | 96 |
| HASPIN | 100 |
| HCK | 5.5 |
| HIPK1 | 21 |
| HIPK2 | 16 |
| HIPK3 | 18 |
| HIPK4 | 36 |
| HPK1 | 32 |
| HUNK | 86 |
| ICK | 79 |
| IGF1R | 95 |
| IKK-alpha | 50 |
| IKK-beta | 90 |
| IKK-epsilon | 98 |
| INSR | 100 |
| INSRR | 81 |
| IRAK1 | 76 |
| IRAK3 | 93 |
| IRAK4 | 81 |
| ITK | 98 |
| JAK1(JH1domain- | 62 |
| catalytic) | |
| JAK1(JH2domain- | 100 |
| pseudokinase) | |
| JAK2(JH1domain- | 84 |
| catalytic) | |
| JAK3(JH1domain- | 45 |
| catalytic) | |
| JNK1 | 28 |
| JNK2 | 0.35 |
| JNK3 | 52 |
| KIT | 12 |
| KIT(A829P) | 12 |
| KIT(D816H) | 79 |
| KIT(D816V) | 66 |
| KIT(L576P) | 4.5 |
| KIT(V559D) | 7.5 |
| KIT(V559D, T670I) | 16 |
| KIT(V559D, V654A) | 51 |
| KIT-autoinhibited | 93 |
| LATS1 | 100 |
| LATS2 | 100 |
| LCK | 2.6 |
| LIMK1 | 96 |
| LIMK2 | 94 |
| LKB1 | 96 |
| LOK | 0.35 |
| LRRK2 | 94 |
| LRRK2(G2019S) | 97 |
| LTK | 84 |
| LYN | 6.5 |
| LZK | 100 |
| MAK | 66 |
| MAP3K1 | 75 |
| MAP3K15 | 70 |
| MAP3K2 | 72 |
| MAP3K3 | 58 |
| MAP3K4 | 100 |
| MAP4K2 | 1.8 |
| MAP4K3 | 93 |
| MAP4K4 | 2.6 |
| MAP4K5 | 59 |
| MAPKAPK2 | 84 |
| MAPKAPK5 | 100 |
| MARK1 | 96 |
| MARK2 | 84 |
| MARK3 | 100 |
| MARK4 | 100 |
| MAST1 | 88 |
| MEK1 | 100 |
| MEK2 | 100 |
| MEK3 | 100 |
| MEK4 | 94 |
| MEK5 | 100 |
| MEK6 | 88 |
| MELK | 85 |
| MERTK | 100 |
| MET | 100 |
| MET(M1250T) | 99 |
| MET(Y1235D) | 96 |
| MINK | 12 |
| MKK7 | 0.9 |
| MKNK1 | 98 |
| MKNK2 | 74 |
| MLCK | 96 |
| MLK1 | 94 |
| MLK2 | 100 |
| MLK3 | 97 |
| MRCKA | 96 |
| MRCKB | 85 |
| MST1 | 100 |
| MST1R | 100 |
| MST2 | 93 |
| MST3 | 88 |
| MST4 | 75 |
| MTOR | 100 |
| MUSK | 48 |
| MYLK | 100 |
| MYLK2 | 100 |
| MYLK4 | 91 |
| MYO3A | 14 |
| MYO3B | 70 |
| NDR1 | 99 |
| NDR2 | 91 |
| NEK1 | 100 |
| NEK10 | 88 |
| NEK11 | 99 |
| NEK2 | 94 |
| NEK3 | 99 |
| NEK4 | 74 |
| NEK5 | 93 |
| NEK6 | 82 |
| NEK7 | 100 |
| NEK9 | 94 |
| NIK | 95 |
| NIM1 | 98 |
| NLK | 49 |
| OSR1 | 62 |
| p38-alpha | 0 |
| p38-beta | 1.4 |
| p38-delta | 55 |
| p38-gamma | 1.3 |
| PAK1 | 87 |
| PAK2 | 90 |
| PAK3 | 78 |
| PAK4 | 100 |
| PAK6 | 93 |
| PAK7 | 98 |
| PCTK1 | 93 |
| PCTK2 | 56 |
| PCTK3 | 76 |
| PDGFRA | 43 |
| PDGFRB | 6.8 |
| PDPK1 | 89 |
| PFCDPK1(P. falciparum) | 1.8 |
| PFPK5(P. falciparum) | 97 |
| PFTAIRE2 | 100 |
| PFTK1 | 77 |
| PHKG1 | 95 |
| PHKG2 | 99 |
| PIK3C2B | 100 |
| PIK3C2G | 100 |
| PIK3CA | 100 |
| PIK3CA(C420R) | 100 |
| PIK3CA(E542K) | 100 |
| PIK3CA(E545A) | 100 |
| PIK3CA(E545K) | 100 |
| PIK3CA(H1047L) | 100 |
| PIK3CA(H1047Y) | 100 |
| PIK3CA(I800L) | 84 |
| PIK3CA(M1043I) | 100 |
| PIK3CA(Q546K) | 91 |
| PIK3CB | 100 |
| PIK3CD | 100 |
| PIK3CG | 100 |
| PIK4CB | 92 |
| PIM1 | 98 |
| PIM2 | 97 |
| PIM3 | 99 |
| PIP5K1A | 90 |
| PIP5K1C | 52 |
| PIP5K2B | 80 |
| PIP5K2C | 76 |
| PKAC-alpha | 93 |
| PKAC-beta | 99 |
| PKMYT1 | 89 |
| PKN1 | 94 |
| PKN2 | 99 |
| PKNB(M. tuberculosis) | 86 |
| PLK1 | 88 |
| PLK2 | 89 |
| PLK3 | 89 |
| PLK4 | 88 |
| PRKCD | 58 |
| PRKCE | 82 |
| PRKCH | 83 |
| PRKCI | 100 |
| PRKCQ | 72 |
| PRKD1 | 100 |
| PRKD2 | 89 |
| PRKD3 | 96 |
| PRKG1 | 97 |
| PRKG2 | 95 |
| PRKR | 85 |
| PRKX | 91 |
| PRP4 | 87 |
| PYK2 | 84 |
| QSK | 87 |
| RAF1 | 99 |
| RET | 29 |
| RET(M918T) | 27 |
| RET(V804L) | 97 |
| RET(V804M) | 65 |
| RIOK1 | 100 |
| RIOK2 | 86 |
| RIOK3 | 93 |
| RIPK1 | 56 |
| RIPK2 | 82 |
| RIPK4 | 90 |
| RIPK5 | 100 |
| ROCK1 | 100 |
| ROCK2 | 82 |
| ROS1 | 100 |
| RPS6KA4(Kin.Dom.1- | 100 |
| N-terminal) | |
| RPS6KA4(Kin.Dom.2- | 94 |
| C-terminal) | |
| RPS6KA5(Kin.Dom.1- | 79 |
| N-terminal) | |
| RPS6KA5(Kin.Dom.2- | 99 |
| C-terminal) | |
| RSK1(Kin.Dom.1- | 83 |
| N-terminal) | |
| RSK1(Kin.Dom.2- | 99 |
| C-terminal) | |
| RSK2(Kin.Dom.1- | 94 |
| N-terminal) | |
| RSK2(Kin.Dom.2- | 100 |
| C-terminal) | |
| RSK3(Kin.Dom.1- | 95 |
| N-terminal) | |
| RSK3(Kin.Dom.2- | 96 |
| C-terminal) | |
| RSK4(Kin.Dom.1- | 90 |
| N-terminal) | |
| RSK4(Kin.Dom.2- | 91 |
| C-terminal) | |
| S6K1 | 100 |
| SBK1 | 100 |
| SGK | 90 |
| SgK110 | 96 |
| SGK2 | 100 |
| SGK3 | 98 |
| SIK | 87 |
| SIK2 | 87 |
| SLK | 63 |
| SNARK | 100 |
| SNRK | 83 |
| SRC | 16 |
| SRMS | 54 |
| SRPK1 | 84 |
| SRPK2 | 86 |
| SRPK3 | 80 |
| STK16 | 93 |
| STK33 | 98 |
| STK35 | 60 |
| STK36 | 39 |
| STK39 | 73 |
| SYK | 24 |
| TAK1 | 0.4 |
| TAOK1 | 14 |
| TAOK2 | 3.7 |
| TAOK3 | 2.8 |
| TBK1 | 100 |
| TEC | 93 |
| TESK1 | 96 |
| TGFBR1 | 100 |
| TGFBR2 | 84 |
| TIE1 | 11 |
| TIE2 | 47 |
| TLK1 | 100 |
| TLK2 | 100 |
| TNIK | 9.7 |
| TNK1 | 93 |
| TNK2 | 100 |
| TNNI3K | 81 |
| TRKA | 82 |
| TRKB | 80 |
| TRKC | 73 |
| TRPM6 | 100 |
| TSSK1B | 99 |
| TTK | 84 |
| TXK | 100 |
| TYK2(JH1domain- | 91 |
| catalytic) | |
| TYK2(JH2domain- | 92 |
| pseudokinase) | |
| TYRO3 | 100 |
| ULK1 | 94 |
| ULK2 | 100 |
| ULK3 | 78 |
| VEGFR2 | 45 |
| VRK2 | 77 |
| WEE1 | 97 |
| WEE2 | 95 |
| WNK1 | 86 |
| WNK3 | 84 |
| YANK1 | 86 |
| YANK2 | 90 |
| YANK3 | 93 |
| YES | 40 |
| YSK1 | 79 |
| YSK4 | 98 |
| ZAK | 3.1 |
| ZAP70 | 72 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound of Formula (I): 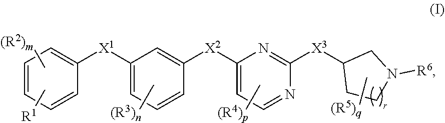
or a pharmaceutically acceptable salt, solvate, hydrate, polymorph, co-crystal, tautomer, stereoisomer, or prodrug thereof, wherein:
R¹ is substituted or unsubstituted C₁-C₆ alkyl;
R², R³, R⁴, and R⁵ are each independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl, —OR^{a}, —N(R^{a})₂, —SR^{a}, —C(═O)R^{a}, —C(═O)OR^{a}, —OC(═O)R^{a}, —C(═O)N(R^{a})₂, —N(R^{a})C(═O)R^{a}, —CN, —SCN, —NO₂;
R⁶ is substituted or unsubstituted alkyl, or substituted or unsubstituted acyl;
X¹ is —O—, —N(R^{b})—, —S—, —C(═O)—, —C(═O)N(R^{b})—, or —N(R^{b})C(═O)—;
X² and X³ are each independently —O—, —N(R^{c})—, or —S—;
each instance of R^{a}, R^{b}, and R^{c} is independently hydrogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;
m, n, p, and q are each independently 0, 1, 2, 3, or 4; and
r is 1, or 2.

2. The compound of claim 1, or a pharmaceutically acceptable salt, solvate, hydrate, polymorph, co-crystal, tautomer, stereoisomer, or prodrug thereof, wherein X² is —O—.

3. The compound of claim 1 or 2, or a pharmaceutically acceptable salt, solvate, hydrate, polymorph, co-crystal, tautomer, stereoisomer, or prodrug thereof, wherein X³ is —N(R^{c})—.

4. The compound of claim 3, or a pharmaceutically acceptable salt, solvate, hydrate, polymorph, co-crystal, tautomer, stereoisomer, or prodrug thereof, wherein X³ is —N(H)—.

5. The compound of any one of claims 1 to 4, wherein the compound is of the Formula (II): 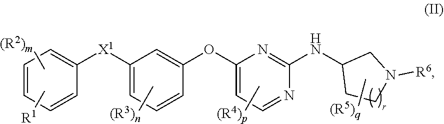
or a pharmaceutically acceptable salt, solvate, hydrate, polymorph, co-crystal, tautomer, stereoisomer, or prodrug thereof.

6. The compound of any one of claims 1 to 5, or a pharmaceutically acceptable salt, solvate, hydrate, polymorph, co-crystal, tautomer, stereoisomer, or prodrug thereof, wherein X¹ is —N(R^{b})C(═O)—.

7. The compound of any one of claims 1 to 6, or a pharmaceutically acceptable salt, solvate, hydrate, polymorph, co-crystal, tautomer, stereoisomer, or prodrug thereof, wherein X¹ is —N(H)C(═O)—.

8. The compound of any one of claims 1 to 7, wherein the compound is of the Formula (III): 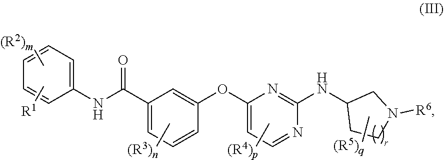
or a pharmaceutically acceptable salt, solvate, hydrate, polymorph, co-crystal, tautomer, stereoisomer, or prodrug thereof.

9. The compound of any one of claims 1 to 8, wherein the compound is of the Formula (IV): 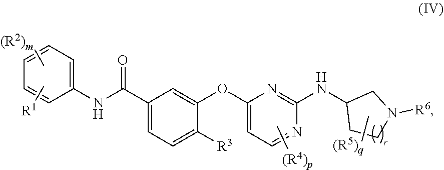
or a pharmaceutically acceptable salt, solvate, hydrate, polymorph, co-crystal, tautomer, stereoisomer, or prodrug thereof.

10. The compound of any one of claims 1 to 9, wherein the compound is of the Formula (V): 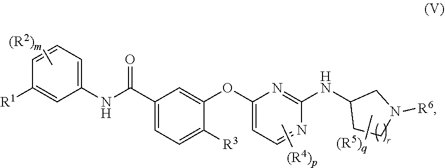
or a pharmaceutically acceptable salt, solvate, hydrate, polymorph, co-crystal, tautomer, stereoisomer, or prodrug thereof.

11. The compound of any one of claims 1 to 10, wherein the compound is of the Formula (VI): 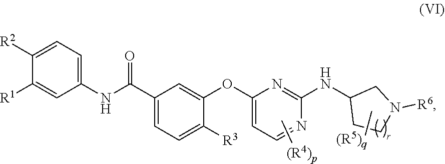
or a pharmaceutically acceptable salt, solvate, hydrate, polymorph, co-crystal, tautomer, stereoisomer, or prodrug thereof.

12. The compound of any one of claims 1 to 10, wherein the compound is of the Formula (VII): 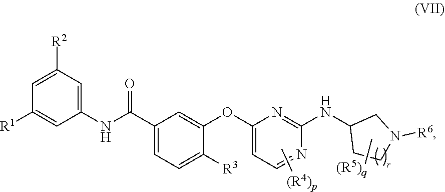
or a pharmaceutically acceptable salt, solvate, hydrate, polymorph, co-crystal, tautomer, stereoisomer, or prodrug thereof.

13. The compound of any one of claims 1 to 12, or a pharmaceutically acceptable salt, solvate, hydrate, polymorph, co-crystal, tautomer, stereoisomer, or prodrug thereof, wherein R¹ is C₁-C₆ alkyl.

14. The compound of any one of claims 1 to 13, or a pharmaceutically acceptable salt, solvate, hydrate, polymorph, co-crystal, tautomer, stereoisomer, or prodrug thereof, wherein R¹ is C₁-C₆ haloalkyl.

15. The compound of any one of claims 1 to 14, or a pharmaceutically acceptable salt, solvate, hydrate, polymorph, co-crystal, tautomer, stereoisomer, or prodrug thereof, wherein R¹ is —CF₃.

16. The compound of any one of claims 1 to 15, or a pharmaceutically acceptable salt, solvate, hydrate, polymorph, co-crystal, tautomer, stereoisomer, or prodrug thereof, wherein R³ is substituted or unsubstituted alkyl.

17. The compound of any one of claims 1 to 16, or a pharmaceutically acceptable salt, solvate, hydrate, polymorph, co-crystal, tautomer, stereoisomer, or prodrug thereof, wherein R³ is substituted or unsubstituted C₁-C₆ alkyl.

18. The compound of any one of claims 1 to 17, or a pharmaceutically acceptable salt, solvate, hydrate, polymorph, co-crystal, tautomer, stereoisomer, or prodrug thereof, wherein R³ is unsubstituted alkyl C₁-C₆ alkyl.

19. The compound of any one of claims 1 to 18, or a pharmaceutically acceptable salt, solvate, hydrate, polymorph, co-crystal, tautomer, stereoisomer, or prodrug thereof, wherein R³ is unsubstituted methyl.

20. The compound of any one of claims 1 to 11, and 13 to 19, wherein the compound is of the Formula (VIII): 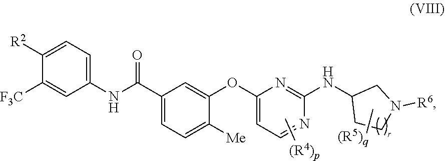
or a pharmaceutically acceptable salt, solvate, hydrate, polymorph, co-crystal, tautomer, stereoisomer, or prodrug thereof.

21. The compound of any one of claims 1 to 10, and 12 to 19, wherein the compound is of the Formula (IX): 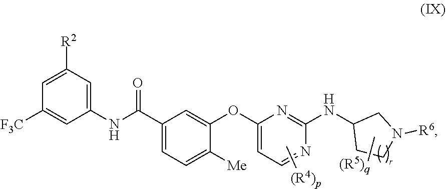
or a pharmaceutically acceptable salt, solvate, hydrate, polymorph, co-crystal, tautomer, stereoisomer, or prodrug thereof.

22. The compound of any one of claims 1 to 21, or a pharmaceutically acceptable salt, solvate, hydrate, polymorph, co-crystal, tautomer, stereoisomer, or prodrug thereof, wherein R⁶ is substituted or unsubstituted acyl.

23. The compound of any one of claims 1 to 20, or a pharmaceutically acceptable salt, solvate, hydrate, polymorph, co-crystal, tautomer, stereoisomer, or prodrug thereof, wherein R⁶ is

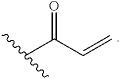 [SMILES: *C(=O)C=C]

24. The compound of any one of claims 1 to 20, or a pharmaceutically acceptable salt, solvate, hydrate, polymorph, co-crystal, tautomer, stereoisomer, or prodrug thereof, wherein R⁶ is

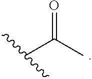 [SMILES: *C(C)=O]

25. The compound of any one of claims 1 to 11, 13 to 20, 22, and 23, wherein the compound is of the Formula (X): 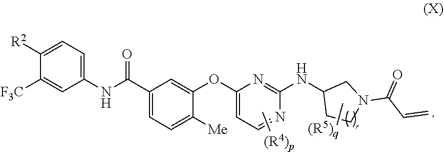
or a pharmaceutically acceptable salt, solvate, hydrate, polymorph, co-crystal, tautomer, stereoisomer, or prodrug thereof.

26. The compound of any one of claims 1 to 10, 12 to 19, and 21 to 23, wherein the compound is of the Formula (XI): 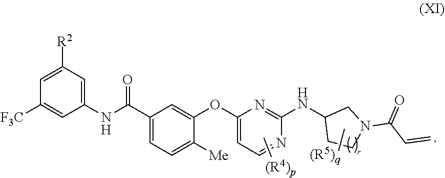
or a pharmaceutically acceptable salt, solvate, hydrate, polymorph, co-crystal, tautomer, stereoisomer, or prodrug thereof.

27. The compound of any one of claims 1 to 26, or a pharmaceutically acceptable salt, solvate, hydrate, polymorph, co-crystal, tautomer, stereoisomer, or prodrug thereof, wherein R² is substituted or unsubstituted alkyl, substituted or unsubstituted heterocyclyl, or substituted or unsubstituted heteroaryl.

28. The compound of any one of claims 1 to 27, or a pharmaceutically acceptable salt, solvate, hydrate, polymorph, co-crystal, tautomer, stereoisomer, or prodrug thereof, wherein R² is substituted or unsubstituted heterocyclyl.

29. The compound of any one of claims 1 to 28, or a pharmaceutically acceptable salt, solvate, hydrate, polymorph, co-crystal, tautomer, stereoisomer, or prodrug thereof, wherein R² is a substituted or unsubstituted piperazine.

30. The compound of any one of claims 1 to 29, or a pharmaceutically acceptable salt, solvate, hydrate, polymorph, co-crystal, tautomer, stereoisomer, or prodrug thereof, wherein R² is of the formula: 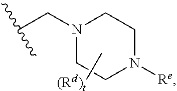
wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;
R^{e} is independently hydrogen, substituted or unsubstituted C₁-C₆ alkyl, substituted or unsubstituted acyl, or a nitrogen protecting group; and
t is 0, 1, 2, 3, 4, 5, 6, 7, or 8.

31. The compound of any one of claims 1 to 30, or a pharmaceutically acceptable salt, solvate, hydrate, polymorph, co-crystal, tautomer, stereoisomer, or prodrug thereof, wherein R² is of the formula: 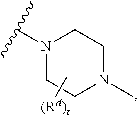
wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl; and
t is 0, 1, 2, 3, 4, 5, 6, 7, or 8.

32. The compound of any one of claims 1 to 31, or a pharmaceutically acceptable salt, solvate, hydrate, polymorph, co-crystal, tautomer, stereoisomer, or prodrug thereof, wherein R² is of the formula:

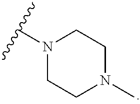 [SMILES: *N1CCN(C)CC1]

33. The compound of any one of claims 1 to 27, or a pharmaceutically acceptable salt, solvate, hydrate, polymorph, co-crystal, tautomer, stereoisomer, or prodrug thereof, wherein R² is substituted or unsubstituted alkyl.

34. The compound of any one of claims 1 to 27, and 33, or a pharmaceutically acceptable salt, solvate, hydrate, polymorph, co-crystal, tautomer, stereoisomer, or prodrug thereof, wherein R² is of the formula: 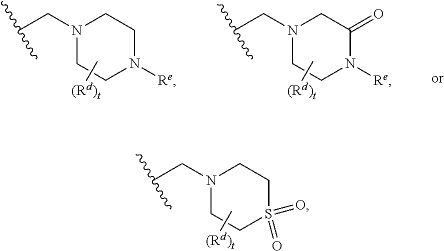
wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;
R^{e} is independently hydrogen, substituted or unsubstituted C₁-C₆ alkyl, substituted or unsubstituted acyl, or a nitrogen protecting group; and
t is 0, 1, 2, 3, 4, 5, 6, 7, or 8.

35. The compound of any one of claims 1 to 27, 33, and 34, or a pharmaceutically acceptable salt, solvate, hydrate, polymorph co-crystal tautomer, stereoisomer, or prodrug thereof, wherein R² is of the formula: 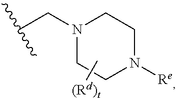
wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;
R^{e} is independently hydrogen, substituted or unsubstituted C₁-C₆ alkyl, substituted or unsubstituted acyl, or a nitrogen protecting group; and
t is 0, 1, 2, 3, 4, 5, 6, 7, or 8.

36. The compound of any one of claims 1 to 27, and 33 to 35, or a pharmaceutically acceptable salt, solvate, hydrate, polymorph, co-crystal, tautomer, stereoisomer, or prodrug thereof wherein R² is of the formula:

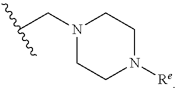

37. The compound of claim 36, wherein R^{e} is substituted or unsubstituted C₁-C₆ alkyl.

38. The compound of claim 37, wherein R^{e} is methyl.

39. The compound of claim 37, wherein R^{e} is ethyl.

40. The compound of any one of claims 1 to 27, and 33 to 34, or a pharmaceutically acceptable salt, solvate, hydrate, polymorph, co-crystal, tautomer, stereoisomer, or prodrug thereof, wherein R² is of the formula: 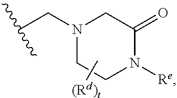
wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl;
R^{e} is independently hydrogen, substituted or unsubstituted C₁-C₆ alkyl, substituted or unsubstituted acyl, or a nitrogen protecting group; and
t is 0, 1, 2, 3, 4, 5, 6, 7, or 8.

41. The compound of any one of claims 1 to 27, 33 to 34, and 40, or a pharmaceutically acceptable salt, solvate, hydrate, polymorph, co-crystal, tautomer, stereoisomer, or prodrug thereof wherein R² is of the formula:

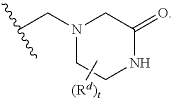

42. The compound of any one of claims 1 to 27, 33 to 34, 36, and 37, or a pharmaceutically acceptable salt, solvate, hydrate, polymorph, co-crystal, tautomer, stereoisomer, or prodrug thereof, wherein R² is of the formula:

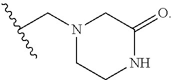 [SMILES: *CN1CCNC(=O)C1]

43. The compound of any one of claims 1 to 27, and 33 to 34, or a pharmaceutically acceptable salt, solvate, hydrate, polymorph, co-crystal, tautomer, stereoisomer, or prodrug thereof, wherein R² is of the formula: 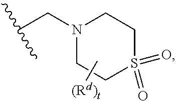
wherein each instance of R^{d} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl; and
t is 0, 1, 2, 3, 4, 5, 6, 7, or 8.

44. The compound of any one of claims 1 to 27, 33 to 34, and 43, or a pharmaceutically acceptable salt, solvate, hydrate, polymorph, co-crystal, tautomer, stereoisomer, or prodrug thereof wherein R² is of the formula:

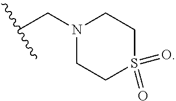 [SMILES: *CN1CCS(=O)(=O)CC1]

45. The compound of any one of claims 1 to 27, or a pharmaceutically acceptable salt, solvate, hydrate, polymorph, co-crystal, tautomer, stereoisomer, or prodrug thereof, wherein R² is substituted or unsubstituted heteroaryl.

46. The compound of any one of claims 1 to 27, and 45, or a pharmaceutically acceptable salt, solvate, hydrate, polymorph, co-crystal, tautomer, stereoisomer, or prodrug thereof, wherein R² is of the formula: 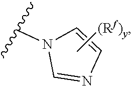
wherein R^{f} is independently hydrogen, halogen, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, or substituted or unsubstituted heteroaryl; and
y is 0, 1, 2, or 3.

47. The compound of any one of claims 1 to 27, 45, and 46, or a pharmaceutically acceptable salt, solvate, hydrate, polymorph, co-crystal, tautomer, stereoisomer, or prodrug thereof, wherein R² is of the formula:

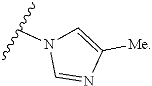 [SMILES: *n1cnc(C)c1]

48. The compound of any one of claims 1 to 47, or a pharmaceutically acceptable salt, solvate, hydrate, polymorph, co-crystal, tautomer, stereoisomer, or prodrug thereof, wherein n is 1.

49. The compound of any one of claims 1 to 48, or a pharmaceutically acceptable salt, solvate, hydrate, polymorph, co-crystal, tautomer, stereoisomer, or prodrug thereof, wherein R⁴ is H.

50. The compound of any one of claims 1 to 49, or a pharmaceutically acceptable salt, solvate, hydrate, polymorph, co-crystal, tautomer, stereoisomer, or prodrug thereof, wherein R⁵ is H.

51. The compound of any one of the preceding claims, of the formula: 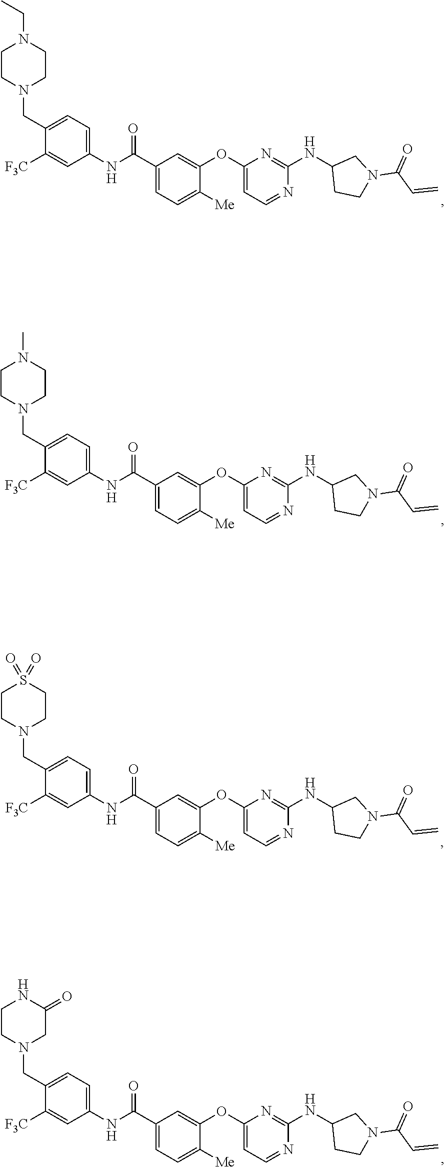 [SMILES: C=CC(=O)N1CCC(Nc2nccc(Oc3cc(C(=O)Nc4ccc(CN5CCN(CC)CC5)c(C(F)(F)F)c4)ccc3C)n2)C1 ; C=CC(=O)N1CCC(Nc2nccc(Oc3cc(C(=O)Nc4ccc(CN5CCN(C)CC5)c(C(F)(F)F)c4)ccc3C)n2)C1 ; C=CC(=O)N1CCC(Nc2nccc(Oc3cc(C(=O)Nc4ccc(CN5CCS(=O)(=O)CC5)c(C(F)(F)F)c4)ccc3C)n2)C1 ; C=CC(=O)N1CCC(Nc2nccc(Oc3cc(C(=O)Nc4ccc(CN5CCNC(=O)C5)c(C(F)(F)F)c4)ccc3C)n2)C1] 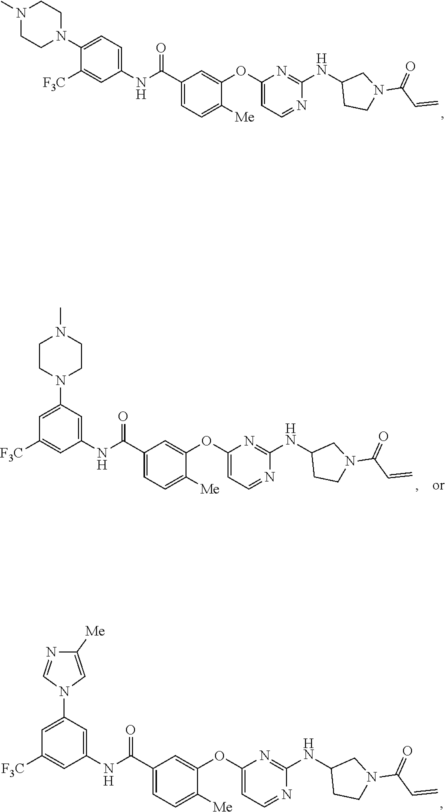 [SMILES: C=CC(=O)N1CCC(Nc2nccc(Oc3cc(C(=O)Nc4ccc(N5CCN(C)CC5)c(C(F)(F)F)c4)ccc3C)n2)C1 ; C=CC(=O)N1CCC(Nc2nccc(Oc3cc(C(=O)Nc4cc(N5CCN(C)CC5)cc(C(F)(F)F)c4)ccc3C)n2)C1 ; C=CC(=O)N1CCC(Nc2nccc(Oc3cc(C(=O)Nc4cc(-n5cnc(C)c5)cc(C(F)(F)F)c4)ccc3C)n2)C1]
or a pharmaceutically acceptable salt, solvate, hydrate, polymorph, co-crystal, tautomer, stereoisomer, or prodrug thereof.

52. The compound of any one of the preceding claims, of the Formula S-(I-1): 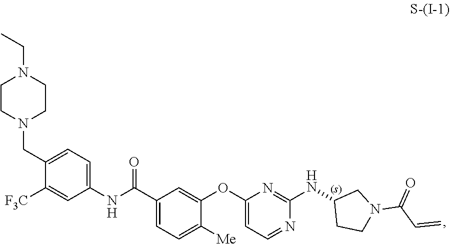
or a pharmaceutically acceptable salt, solvate, hydrate, polymorph, co-crystal, tautomer, stereoisomer, isotopically labeled derivative, or prodrug thereof.

53. The compound of any one of the preceding claims, of the Formula R-(I-1): 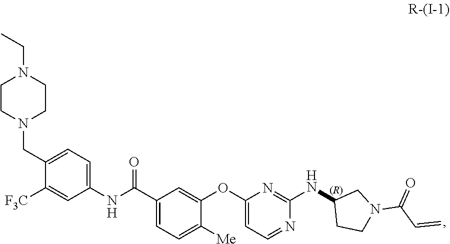
or a pharmaceutically acceptable salt, solvate, hydrate, polymorph, co-crystal, tautomer, stereoisomer, isotopically labeled derivative, or prodrug thereof.

54. The compound of any one of the preceding claims, of the formula: 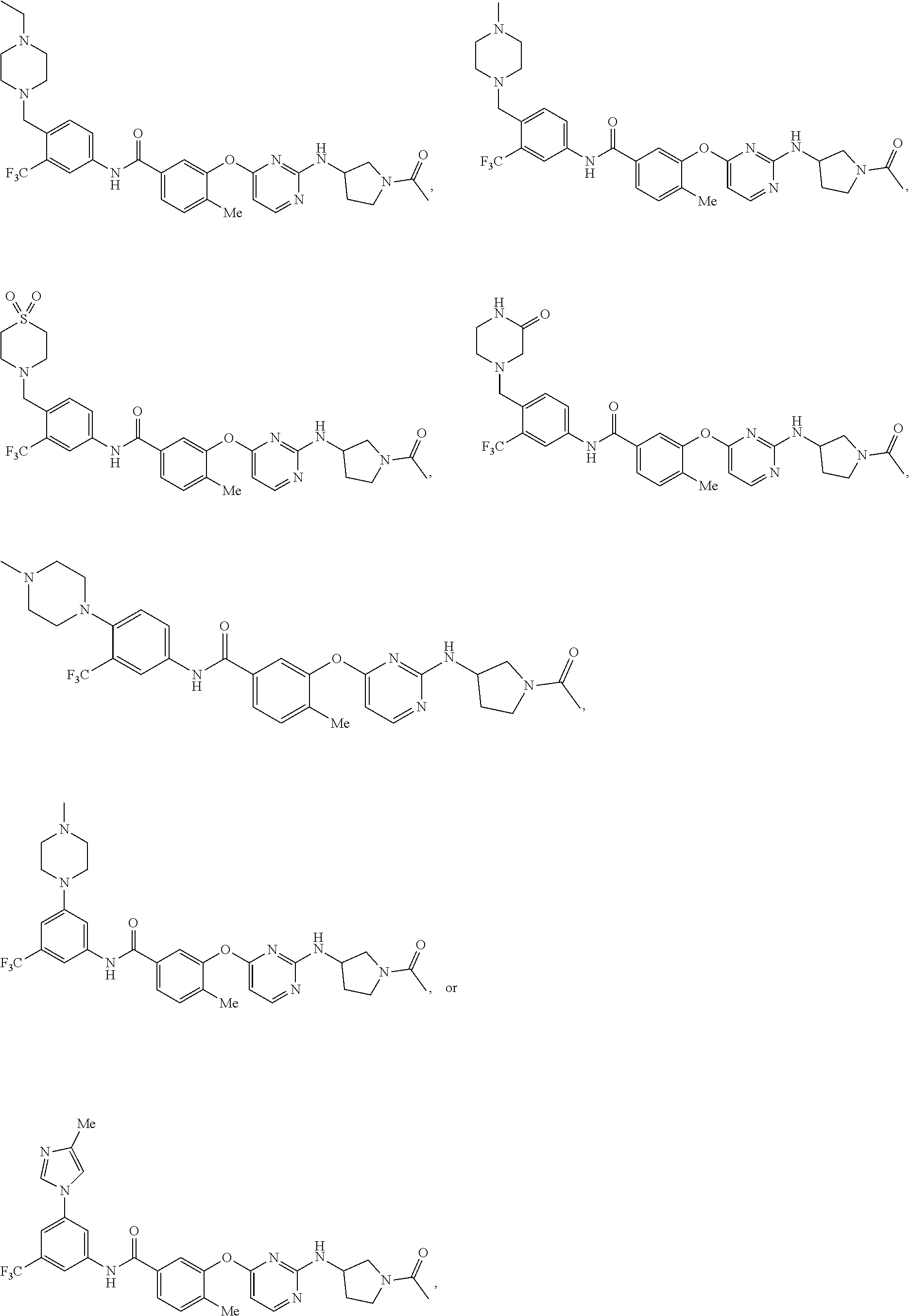
or a pharmaceutically acceptable salt, solvate, hydrate, polymorph, co-crystal, tautomer, stereoisomer, or prodrug thereof.

55. The compound of any one of claims 1 to 54, or a pharmaceutically acceptable salt thereof.

56. A pharmaceutical composition comprising a compound of any one of claims 1 to 55, or a pharmaceutically acceptable salt, solvate, hydrate, polymorph, co-crystal, tautomer, stereoisomer, isotopically labeled derivative, or prodrug thereof; and optionally a pharmaceutically acceptable excipient.

57. The pharmaceutical composition of claim 56, further comprising an additional pharmaceutical agent.

58. A method of treating a disease in a subject in need thereof, the method comprising administering to the subject in need thereof a therapeutically effective amount of a compound of any one claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57.

59. The method of claim 58, wherein the disease is a viral infection.

60. The method of claim 58 or 59, wherein the disease is a viral infection resulting from a hepatitis virus.

61. The method of claim 58 or 59, wherein the disease is a viral infection resulting from hepatitis B.

62. The method of claim 58 or 59, wherein the disease is a viral infection resulting from a flavivirus.

63. The method of claim 58, 59, or 62, wherein the disease is a viral infection resulting from Zika virus.

64. The method of claim 58, wherein the disease is a neurological disorder.

65. The method of claim 64, wherein the neurological disorder is Alzheimer's disease.

66. The method of claim 58, where in the disease is a proliferative disease.

67. The method of claim 66, wherein the proliferative disease is cancer.

68. The method of claim 67, wherein the cancer is lung cancer, prostate cancer, skin cancer, thyroid cancer, pancreatic cancer, colorectal cancer, liver cancer, leukemia, or lymphoma.

69. A method of treating an inflammatory disease in a subject in need thereof, the method comprising administering to the subject in need thereof a therapeutically effective amount of a compound of any one claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57.

70. The method of any one of claim 58 to 69, wherein the disease is a disease associated with a mitogen-activated protein kinase kinase (MAP2K).

71. The method of any one of claims 58 to 70, wherein the disease is a disease associated with higher-than-normal activity of a mitogen-activated protein kinase kinase (MAP2K).

72. A method of inhibiting the activity of a mitogen-activated protein kinase kinase (MAP2K) in a subject in need thereof, the method comprising administering to the subject in need thereof an effective amount of a compound of any one of claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57.

73. The method of any one of claims 58 to 72, wherein the subject is a human.

74. A method of inhibiting the activity of a mitogen-activated protein kinase kinase (MAP2K) in a cell, the method comprising contacting the cell with an effective amount of a compound of any one claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57.

75. The method of any one claims 58 to 74, wherein the MAP2K is Dual specificity mitogen-activated protein kinase kinase 7 (MKK7).

76. A method of inhibiting the activity of a c-Jun N-terminal kinase (JNK) in a subject in need thereof, the method comprising administering to the subject in need thereof an effective amount of a compound of any one of claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57.

77. The method of claim 76, wherein the JNK is JNK1.

78. The method of claim 77, wherein the JNK is JNK2.

79. A compound of any one of claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57, for use in the treatment of a disease in a subject in need thereof.

80. A compound of any one of claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57, for use in the manufacture of a medicament for the treatment of diseases.

81. Use of a compound of any one of claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57, in the treatment of a disease in a subject in need thereof.

82. A kit comprising:
a compound of any one of claims 1 to 55, or a pharmaceutically acceptable salt, solvate, hydrate, polymorph, co-crystal, tautomer, stereoisomer, isotopically labeled derivative, or prodrug thereof, or a pharmaceutical composition of claim 56 or 57; and instructions for using the compound, or a pharmaceutically acceptable salt, solvate, hydrate, polymorph, co-crystal, tautomer, stereoisomer, isotopically labeled derivative, or prodrug thereof, or the pharmaceutical composition.
